SEQUENCE LISTING (19 sequences)

SEQ ID NO: 1 - protein, 217 aa, unidentified
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 121 aa, synthetic construct
QIQLVQSGPELKNPGETVTISCKASGYTFTTYGLTWVKQAPGKGLKWMGWINTYSGVPTHADDFTGRFAFSLETSASTAYLQIDNLNNEDTATYFCARTYGERRDYSMDYWGQGTSVTVSS

SEQ ID NO: 3 - protein, 111 aa, synthetic construct
DIVFTQSPASLAVSLGQRATISCRASESVEYYGTSLMQWFQQKPGQPPKLLIYAASNVESGVPARFSGSGSGTDFSLNIHPVEEDDMAMYFCQQSRKVPWTFGGGTKLEIK

SEQ ID NO: 4 - protein, 8 aa, synthetic construct
GYTFTTYG

SEQ ID NO: 5 - protein, 8 aa, synthetic construct
INTYSGVP

SEQ ID NO: 6 - protein, 14 aa, synthetic construct
ARTYGERRDYSMDY

SEQ ID NO: 7 - protein, 10 aa, synthetic construct
ESVEYYGTSL

SEQ ID NO: 8 - skipped

SEQ ID NO: 9 - protein, 9 aa, synthetic construct
QQSRKVPWT

SEQ ID NO: 10 - protein, 14 aa, synthetic construct
  SITE 7: note = Phosphorylated residue
RTPSLPTPPTREPK

SEQ ID NO: 11 - protein, 14 aa, synthetic construct
RTPSLPTPPTREPK

SEQ ID NO: 12 - protein, 25 aa, synthetic construct
QIQLVQSGPELKNPGETVTISCKAS

SEQ ID NO: 13 - protein, 17 aa, synthetic construct
LTWVKQAPGKGLKWMGW

SEQ ID NO: 14 - protein, 38 aa, synthetic construct
THADDFTGRFAFSLETSASTAYLQIDNLNNEDTATYFC

SEQ ID NO: 15 - protein, 11 aa, synthetic construct
WGQGTSVTVSS

SEQ ID NO: 16 - protein, 26 aa, synthetic construct
DIVFTQSPASLAVSLGQRATISCRAS

SEQ ID NO: 17 - protein, 17 aa, synthetic construct
MQWFQQKPGQPPKLLIY

SEQ ID NO: 18 - protein, 36 aa, synthetic construct
NVESGVPARFSGSGSGTDFSLNIHPVEEDDMAMYFC

SEQ ID NO: 19 - protein, 10 aa, synthetic construct
FGGGTKLEIK

(19) United States
(12) Patent Application Publication
(10) Pub. No.: US 2026/0079168 A1
(43) Pub. Date: Mar. 19, 2026

(54) TAU PHOSPHO (THR217) BINDING ANTIBODIES AND ANTIGEN BINDING FRAGMENTS THEREOF

(71) Applicant: BioLegend, Inc., San Diego, CA (US)

(72) Inventors: Efthalia CHRONOPOULOU, San Diego, CA (US); Patricia Hiroko TANI, San Diego, CA (US); Giovanni SUAREZ, San Diego, CA (US); Joseph Corey EVANS, San Diego, CA (US)

(21) Appl. No.: 19/329,424

(22) Filed: Sep. 15, 2025

Related U.S. Application Data
(60) Provisional application No. 63/696,729, filed on Sep. 19, 2024.

(51) Int. Cl. G01N 33/68; C07K 16/18

(52) U.S. Cl. CPC G01N 33/6896; C07K 16/18; C07K 2317/33; C07K 2317/92

(57) ABSTRACT

The technology relates in part to antibodies or antigen-binding fragments thereof that bind Tau phosphorylated at threonine 217 (Tau phospho (Thr217)) or a portion thereof, as well as methods, systems and kits for detection of Tau phospho (Thr217). In certain aspects, the technology relates to antibodies or antigen binding fragments thereof for use in determining levels of Tau phospho (Thr217) in a sample containing or suspected of containing Tau phospho (Thr217). In some aspects, the technology relates to antibodies or antigen-binding fragments thereof for use in diagnosing or treating an individual with or suspected of having a disease or disorder associated with Tau phospho (Thr217).

### RELATED PATENT APPLICATIONS

This patent application claims the benefit of U.S. provisional patent application No. 63/696,729 filed on Sep. 19, 2024, entitled TAU PHOSPHO (THR217) BINDING ANTIBODIES AND ANTIGEN BINDING FRAGMENTS THEREOF, naming Efthalia CHRONOPOULOU et al. as inventors, and designated by attorney docket no. BIOLEG-1003PROV. The entire content of the foregoing patent application is incorporated herein by reference for all purposes, including all text, tables and drawings.

### SEQUENCE LISTING

The instant application contains a Sequence Listing which has been submitted electronically in XML format and is hereby incorporated by reference in its entirety. Said XML copy, created on Sep. 4, 2025, is named BIOLEG-1003_SL.xml and is 17,531 bytes in size.

### FIELD

The technology relates in part to antibodies or antigen-binding fragments thereof that bind Tau phosphorylated at threonine 217 (Tau phospho (Thr217)) or a portion thereof, as well as methods, systems and kits for detection of Tau phospho (Thr217). In certain aspects, the technology relates to antibodies or antigen binding fragments thereof for use in determining levels of Tau phospho (Thr217) in a sample containing or suspected of containing Tau phospho (Thr217). In some aspects, the technology relates to antibodies or antigen-binding fragments thereof for use in diagnosing or treating an individual with or suspected of having a disease or disorder associated with Tau phospho (Thr217).

### BACKGROUND

Tau (tubulin associated unit) proteins form a group of six soluble protein isoforms produced by alternative splicing from the gene MAPT (microtubule-associated protein tau). Tau proteins typically maintain stability of microtubules in axons and are abundant in neurons of the central nervous system (CNS; e.g., cerebral cortex). Tau proteins also are expressed in astrocytes, glial cells, and oligodendrocytes. Pathologies and dementias of the nervous system such as Alzheimer's disease (AD) and Parkinson's disease (PD) are often associated with tau proteins that have become hyperphosphorylated insoluble aggregates referred to as neurofibrillary tangles.

Phosphorylation of Tau at Thr217 is a biomarker for stages of AD and can be detected years prior to the appearance of AD symptoms. Tau phospho (Thr217) can be 3-6× more phosphorylated in cerebrospinal fluid (CSF) samples from AD patients compared to either control subjects or subjects with mild cognitive impairment. Also, the level of Tau phospho (Thr217) increases with AD progression (i.e., the phosphorylation level is positively correlated with Braak staging, which is a common method to classify AD severity). Developing tools to build diagnostic assays that can quantitate the levels of Tau phospho (Thr217) with specificity and sensitivity in biological fluids could help to both accurately identify individuals with preclinical signs of AD and further help to monitor their disease progression following treatment.

Existing technologies for diagnosis of AD rely on biomarker detection in cerebrospinal fluid (CSF) and plasma. Current biomarker profiles used for AD diagnosis include measuring the ratio of amyloid beta (1-42) to amyloid beta (1-40), which can be an accurate diagnostic for disease severity. In addition, the levels of total Tau and Tau phospho (Thr181) are increased. However, changes of total Tau and Tau phospho (Thr181) are not AD-specific and are observed in other tauopathies, such as progressive supranuclear palsy, frontotemporal lobar degeneration, and some other forms of dementia. Tau phospho (Thr217), however, constitutes a highly specific marker to detect both preclinical and advanced AD stages. It is desirable to have highly specific antibodies that can be used to develop quantitative assays that measure the levels of Tau phospho (Thr217) in biological fluids. Ideally, these assays should be sensitive enough to detect <0.07 pg/ml of phosphorylated Tau in serum, plasma, and CSF.

Provided herein are highly specific antibodies that can be used to detect Tau phospho (Thr217) by a variety of methods (e.g. Western blot (WB), immunohistochemistry (IHC), enzyme-linked immunosorbent assay (ELISA), immunoassay, and the like) in both animal models where Tau phospho (Thr217) is differentially phosphorylated and brain samples from AD patients. These high affinity antibodies can be used for developing diagnostic assays for preclinical and advanced forms of AD, for example. Currently available Tau phospho (Thr217) antibodies and associated methods are limited in their range of both in vitro and in vivo applications. Thus, there remains a need for reagents and methods for detecting Tau phospho (Thr217). Provided herein are embodiments that meet such need.

### SUMMARY

Provided in certain aspects are isolated antibodies or antigen binding fragments thereof that bind tubulin associated unit phosphorylated at threonine 217 (Tau phospho (Thr217)) or a portion thereof, comprising (a) an immunoglobulin heavy chain variable domain comprising (i) a heavy chain complementarity determining region 1 (CDRH1) comprising the polypeptide GYTFTTYG (SEQ ID NO: 4), or a polypeptide that is at least 80 percent identical to the polypeptide GYTFTTYG (SEQ ID NO: 4); (ii) a heavy chain complementarity determining region 2 (CDRH2) comprising the polypeptide INTYSGVP (SEQ ID NO: 5), or a polypeptide that is at least 80 percent identical to the polypeptide INTYSGVP (SEQ ID NO: 5); and (iii) a heavy chain complementarity determining region 3 (CDRH3) comprising the polypeptide ARTYGERRDYSMDY (SEQ ID NO: 6), or a polypeptide that is at least 80 percent identical to the polypeptide ARTYGERRDYSMDY (SEQ ID NO: 6); and (b) an immunoglobulin light chain variable domain comprising (i) a light chain complementarity determining region 1 (CDRL1) comprising the polypeptide ESVEYYGTSL (SEQ ID NO: 7), or a polypeptide that is at least 80 percent identical to the polypeptide ESVEYYGTSL (SEQ ID NO: 7); (ii) a light chain complementarity determining region 2 (CDRL2) comprising the polypeptide AAS, or a polypeptide that is at least 80 percent identical to the polypeptide AAS; and (iii) a light chain complementarity determining region 3 (CDRL3) comprising the polypeptide QQSRKVPWT (SEQ ID NO: 9), or a polypeptide that is at least 80 percent identical to the polypeptide QQSRKVPWT (SEQ ID NO: 9).

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows clone screening of purified antibodies by Western blot. Panel A: whole cell extracts from rat brain (day 2 post birth). Panel B: whole cell extracts from rat brain (day 30 post birth). Lane 1: molecular weight markers. Lane 2: anti-Actin (BD #612656 at 1:3,000). Lane 3: commercial anti-Tau pan antibody at 1:10,000. Lane 4: anti-Tau phospho (Thr217) commercial benchmark antibody (AB1) control at 1:7,500. Lanes 5-8: anti-Tau phospho (Thr217) Clone A at 1 μg/ml, 0.5 μg/ml, 0.25 μg/ml, and 0.125 μg/ml. Lanes 9-12: anti-Tau phospho (Thr217) Clone B at 1 μg/ml, 0.5 μg/ml, 0.25 μg/ml, and 0.125 μg/ml. Lane 13: commercial pan anti-Tau antibody at 1:10,000.

FIG. 2 shows clone screening by direct ELISA using biotinylated non-phospho and phospho (Thr217) Tau peptides. Direct ELISA of anti-Tau phospho (Thr217) Antibody (Clone A, top plot; and Clone B, bottom plot) was performed.

FIG. 3 shows Tau phospho (Thr217) antibody verification by sandwich ELISA. Performance of Clone A and Clone B in sandwich ELISA were compared to a Tau phospho (Thr217)-specific polyclonal antibody.

FIG. 4 shows Tau phospho (Thr217) antibody verification by sandwich ELISA. Cross-reactivity with other Tau phospho sites is shown.

FIG. 5 shows Tau phospho (Thr217) antibody verification by Western blot. P5: whole cell rat brain extracts (15 μg total protein) from 5 days old (positive control). P31: whole cell rat brain extracts (15 μg total protein) from 31 days old (negative control). Left panel: purified anti-Tau phospho (Thr217) (Clone B). Middle panel: purified anti-Tau phospho (Thr217) (commercial benchmark antibody (AB2)). Right panel: purified commercial pan anti-Tau antibody. Lane M: molecular weight marker.

FIG. 6 shows Tau phospho (Thr217) antibody verification by Western blot. P2: whole cell rat brain extracts (15 μg total protein) from 2 days old (positive control). P31: whole cell rat brain extracts (15 μg total protein) from 31 days old (negative control). Lane M: Molecular weight marker.

FIG. 7 shows Tau phospho (Thr217) antibody verification by IHC. Top panels: formalin-fixed paraffin-embedded human Alzheimer's disease cortex (positive control). Bottom panels: formalin-fixed paraffin-embedded human normal cortex (negative control). Left panels: tissue sections were incubated with Alexa Fluor® 647 secondary antibody only. Middle panels: tissue sections were incubated with purified anti-Tau phospho (Thr217) (Clone B). Right panels: tissue sections were incubated with purified commercial benchmark antibody (AB3).

FIG. 8 shows Tau phospho (Thr217) antibody verification by IHC-P (phospho specificity verification). Panel B: formalin-fixed paraffin-embedded human Alzheimer's disease cortex untreated (positive control, LPP−). Panel C: formalin-fixed paraffin-embedded human Alzheimer's disease cortex treated with Lambda protein phosphatase (LPP) (negative control, LPP+). Panel A: tissue sections incubated with Alexa Fluor® 647 secondary antibody only. Panels B and C: tissue sections incubated with purified anti-Tau phospho (Thr217) (Clone B). Panel D represents the signal to noise ratio (S/N ratio) (mean±SEM) of LPP untreated tissue (LPP−) versus LPP treated tissue (LPP+) (*: p=0.0207).

### DETAILED DESCRIPTION

Provided herein are tubulin associated unit phosphorylated at threonine 217 (Tau phospho (Thr217)) binding antibodies and antigen binding fragments thereof, nucleic acids encoding such antibodies, components thereof, and antigen-binding fragments thereof. Also provided herein are vectors (e.g., recombinant expression vectors) and cells (e.g., recombinant cells) for expression and production of the antibodies and antigen-binding fragments. In some embodiments, antibodies and antigen binding fragments thereof can bind to Tau phospho (Thr217) under physiological and/or in vitro conditions. Also provided are methods of producing and using Tau phospho (Thr217) binding antibodies and antigen-binding fragments thereof such as in methods for detecting Tau phospho (Thr217) in a sample from an individual, including methods for laboratory/research purposes (e.g., flow cytometry, ELISA, and/or Western blot), and/or for the use and treatment and/or prevention of various diseases or disorders through the delivery of pharmaceutical or other compositions that contain such antibodies or antigen-binding fragments thereof.

### Anti-Tau Phospho (Thr217) Agents

Provided herein are agents that bind tubulin associated unit phosphorylated at threonine 217 (Tau phospho (Thr217)) or a portion thereof. Agents that bind Tau phospho (Thr217) or a portion thereof may be referred to as anti-Tau phospho (Thr217) agents and may include anti-Tau phospho (Thr217) antibodies, anti-Tau phospho (Thr217) antibody fragments (e.g., antigen binding fragments), and anti-Tau phospho (Thr217) antibody derivatives. In some embodiments, the agent is isolated (e.g., separated from a component of its natural environment [e.g., an animal, a biological sample]). In some embodiments, the agent is non-naturally occurring (e.g., produced by human intervention). In some embodiments, the agent is a humanized antibody, or an antigen binding fragment thereof. In some embodiments, the agent is a derivative of a humanized antibody that binds Tau phospho (Thr217).

In some embodiments, the agent binds Tau phospho (Thr217) under laboratory conditions (e.g., binds Tau phospho (Thr217) in vitro, binds Tau phospho (Thr217) in a flow cytometry assay, binds Tau phospho (Thr217) in an ELISA). In some embodiments, the agent binds Tau phospho (Thr217) under physiological conditions (e.g., binds Tau phospho (Thr217) in a cell in a subject).

Generally, an anti-Tau phospho (Thr217) agent provided herein comprises at least one immunoglobulin heavy chain variable domain and at least one immunoglobulin light chain variable domain. In some embodiments, an anti-Tau phospho (Thr217) agent herein comprises two immunoglobulin heavy chain variable domains and two immunoglobulin light chain variable domains. Typically, each immunoglobulin heavy chain variable domain of the anti-Tau phospho (Thr217) agent comprises first, second, and third heavy chain complementarity determining regions (CDRs; CDRH1, CDRH2, CDRH3), and each immunoglobulin light chain variable domain of the anti-Tau phospho (Thr217) agent comprises first, second, and third light chain CDRs (CDRL1, CDRL2, CDRL3).

In some embodiments, an anti-Tau phospho (Thr217) agent provided herein comprises a heavy chain complementarity determining region 1 (CDRH1) comprising the polypeptide GYTFTTYG (SEQ ID NO: 4), or a polypeptide that is at least 80 percent identical to the polypeptide GYTFTTYG (SEQ ID NO: 4). In some embodiments, the anti-Tau phospho (Thr217) agent provided herein comprises a CDRH1 comprising a polypeptide that is at least 85 percent identical to the polypeptide GYTFTTYG (SEQ ID NO: 4). In some embodiments, the anti-Tau phospho (Thr217) agent provided herein comprises a CDRH1 comprising a polypeptide that is at least 90 percent identical to the polypeptide GYTFTTYG (SEQ ID NO: 4). In some embodiments, the anti-Tau phospho (Thr217) agent provided herein comprises a CDRH1 comprising a polypeptide that is at least 95 percent identical to the polypeptide GYTFTTYG (SEQ ID NO: 4). In some embodiments, the anti-Tau phospho (Thr217) agent provided herein comprises a CDRH1 comprising a polypeptide that is 100 percent identical to the polypeptide GYTFTTYG (SEQ ID NO: 4).

In some embodiments, an anti-Tau phospho (Thr217) agent provided herein comprises a heavy chain complementarity determining region 2 (CDRH2) comprising the polypeptide INTYSGVP (SEQ ID NO: 5), or a polypeptide that is at least 80 percent identical to the polypeptide INTYSGVP (SEQ ID NO: 5). In some embodiments, the anti-Tau phospho (Thr217) agent provided herein comprises a CDRH2 comprising a polypeptide that is at least 85 percent identical to the polypeptide INTYSGVP (SEQ ID NO: 5). In some embodiments, the anti-Tau phospho (Thr217) agent provided herein comprises a CDRH2 comprising a polypeptide that is at least 90 percent identical to the polypeptide INTYSGVP (SEQ ID NO: 5). In some embodiments, the anti-Tau phospho (Thr217) agent provided herein comprises a CDRH2 comprising a polypeptide that is at least 95 percent identical to the polypeptide INTYSGVP (SEQ ID NO: 5). In some embodiments, the anti-Tau phospho (Thr217) agent provided herein comprises a CDRH2 comprising a polypeptide that is 100 percent identical to the polypeptide INTYSGVP (SEQ ID NO: 5).

In some embodiments, an anti-Tau phospho (Thr217) agent provided herein comprises a heavy chain complementarity determining region 3 (CDRH3) comprising the polypeptide ARTYGERRDYSMDY (SEQ ID NO: 6), or a polypeptide that is at least 80 percent identical to the polypeptide ARTYGERRDYSMDY (SEQ ID NO: 6). In some embodiments, the anti-Tau phospho (Thr217) agent provided herein comprises a CDRH3 comprising a polypeptide that is at least 85 percent identical to the polypeptide ARTYGERRDYSMDY (SEQ ID NO: 6). In some embodiments, the anti-Tau phospho (Thr217) agent provided herein comprises a CDRH3 comprising a polypeptide that is at least 90 percent identical to the polypeptide ARTYGERRDYSMDY (SEQ ID NO: 6). In some embodiments, the anti-Tau phospho (Thr217) agent provided herein comprises a CDRH3 comprising a polypeptide that is at least 95 percent identical to the polypeptide ARTYGERRDYSMDY (SEQ ID NO: 6). In some embodiments, the anti-Tau phospho (Thr217) agent provided herein comprises a CDRH3 comprising a polypeptide that is at 100 percent identical to the polypeptide ARTYGERRDYSMDY (SEQ ID NO: 6).

In some embodiments, an anti-Tau phospho (Thr217) agent provided herein comprises a light chain complementarity determining region 1 (CDRL1) comprising the polypeptide ESVEYYGTSL (SEQ ID NO: 7), or a polypeptide that is at least 80 percent identical to the polypeptide ESVEYYGTSL (SEQ ID NO: 7). In some embodiments, the anti-Tau phospho (Thr217) agent provided herein comprises a CDRL1 comprising a polypeptide that is at least 85 percent identical to the polypeptide ESVEYYGTSL (SEQ ID NO: 7). In some embodiments, the anti-Tau phospho (Thr217) agent provided herein comprises a CDRL1 comprising a polypeptide that is at least 90 percent identical to the polypeptide ESVEYYGTSL (SEQ ID NO: 7). In some embodiments, the anti-Tau phospho (Thr217) agent provided herein comprises a CDRL1 comprising a polypeptide that is at least 95 percent identical to the polypeptide ESVEYYGTSL (SEQ ID NO: 7). In some embodiments, the anti-Tau phospho (Thr217) agent provided herein comprises a CDRL1 comprising a polypeptide that is 100 percent identical to the polypeptide ESVEYYGTSL (SEQ ID NO: 7).

In some embodiments, an anti-Tau phospho (Thr217) agent provided herein comprises a light chain complementarity determining region 2 (CDRL2) comprising the polypeptide AAS, or a polypeptide that is at least 80 percent identical to the polypeptide AAS. In some embodiments, the anti-Tau phospho (Thr217) agent provided herein comprises a CDRL2 comprising a polypeptide that is at least 85 percent identical to the polypeptide AAS. In some embodiments, the anti-Tau phospho (Thr217) agent provided herein comprises a CDRL2 comprising a polypeptide that is at least 90 percent identical to the polypeptide AAS. In some embodiments, the anti-Tau phospho (Thr217) agent provided herein comprises a CDRL2 comprising a polypeptide that is at least 95 percent identical to the polypeptide AAS. In some embodiments, the anti-Tau phospho (Thr217) agent provided herein comprises a CDRL2 comprising a polypeptide that is 100 percent identical to the polypeptide AAS.

In some embodiments, an anti-Tau phospho (Thr217) agent provided herein comprises a light chain complementarity determining region 3 (CDRL3) comprising the polypeptide QQSRKVPWT (SEQ ID NO: 9), or a polypeptide that is at least 80 percent identical to the polypeptide QQSRKVPWT (SEQ ID NO: 9). In some embodiments, the anti-Tau phospho (Thr217) agent provided herein comprises a CDRL3 comprising a polypeptide that is at least 85 percent identical to the polypeptide QQSRKVPWT (SEQ ID NO: 9). In some embodiments, the anti-Tau phospho (Thr217) agent provided herein comprises a CDRL3 comprising a polypeptide that is at least 90 percent identical to the polypeptide QQSRKVPWT (SEQ ID NO: 9). In some embodiments, the anti-Tau phospho (Thr217) agent provided herein comprises a CDRL3 comprising a polypeptide that is at least 95 percent identical to the polypeptide QQSRKVPWT (SEQ ID NO: 9). In some embodiments, the anti-Tau phospho (Thr217) agent provided herein comprises a CDRL3 comprising a polypeptide that is at least 100 percent identical to the polypeptide QQSRKVPWT (SEQ ID NO: 9).

In some embodiments, an anti-Tau phospho (Thr217) agent provided herein comprises an immunoglobulin heavy chain variable domain comprising the polypeptide of SEQ ID NO: 2, or a polypeptide that is at least 80 percent identical to the polypeptide of SEQ ID NO: 2. In some embodiments, the anti-Tau phospho (Thr217) agent provided herein comprises an immunoglobulin heavy chain variable domain comprising a polypeptide that is at least 85 percent identical to the polypeptide of SEQ ID NO: 2. In some embodiments, the anti-Tau phospho (Thr217) agent provided herein comprises an immunoglobulin heavy chain variable domain comprising a polypeptide that is at least 90 percent identical to the polypeptide of SEQ ID NO: 2. In some embodiments, the anti-Tau phospho (Thr217) agent provided herein comprises an immunoglobulin heavy chain variable domain comprising a polypeptide that is at least 95 percent identical to the polypeptide of SEQ ID NO: 2. In some embodiments, the anti-Tau phospho (Thr217) agent provided herein comprises an immunoglobulin heavy chain variable domain comprising a polypeptide that is 100 percent identical to the polypeptide of SEQ ID NO: 2.

In some embodiments, an anti-Tau phospho (Thr217) agent provided herein comprises an immunoglobulin light chain variable domain comprising the polypeptide of SEQ ID NO: 3, or a polypeptide that is at least 80 percent identical to the polypeptide of SEQ ID NO: 3. In some embodiments, the anti-Tau phospho (Thr217) agent provided herein comprises an immunoglobulin light chain variable domain comprising a polypeptide that is at least 85 percent identical to the polypeptide of SEQ ID NO: 3. In some embodiments, the anti-Tau phospho (Thr217) agent provided herein comprises an immunoglobulin light chain variable domain comprising a polypeptide that is at least 90 percent identical to the polypeptide of SEQ ID NO: 3. In some embodiments, the anti-Tau phospho (Thr217) agent provided herein comprises an immunoglobulin light chain variable domain comprising a polypeptide that is at least 95 percent identical to the polypeptide of SEQ ID NO: 3. In some embodiments, the anti-Tau phospho (Thr217) agent provided herein comprises an immunoglobulin light chain variable domain comprising a polypeptide that is at least 100 percent identical to the polypeptide of SEQ ID NO: 3.

In some embodiments, an anti-Tau phospho (Thr217) agent provided herein comprises a heavy chain framework region 1 (fwr1) comprising the polypeptide QIQLVQSGPELKNPGETVTISCKAS (SEQ ID NO: 12), or a polypeptide that is at least 80 percent identical to the polypeptide QIQLVQSGPELKNPGETVTISCKAS (SEQ ID NO: 12). In some embodiments, the anti-Tau phospho (Thr217) agent provided herein comprises a heavy chain fwr1 comprising a polypeptide that is at least 85 percent identical to the polypeptide QIQLVQSGPELKNPGETVTISCKAS (SEQ ID NO: 12). In some embodiments, the anti-Tau phospho (Thr217) agent provided herein comprises a heavy chain fwr1 comprising a polypeptide that is at least 90 percent identical to the polypeptide QIQLVQSGPELKNPGETVTISCKAS (SEQ ID NO: 12). In some embodiments, the anti-Tau phospho (Thr217) agent provided herein comprises a heavy chain fwr1 comprising a polypeptide that is at least 95 percent identical to the polypeptide QIQLVQSGPELKNPGETVTISCKAS (SEQ ID NO: 12). In some embodiments, the anti-Tau phospho (Thr217) agent provided herein comprises a heavy chain fwr1 comprising a polypeptide that is at least 100 percent identical to the polypeptide

(SEQ ID NO: 12)
QIQLVQSGPELKNPGETVTISCKAS

In some embodiments, an anti-Tau phospho (Thr217) agent provided herein comprises a heavy chain framework region 2 (fwr2) comprising the polypeptide LTWVKQAPGKGLKWMGW (SEQ ID NO: 13), or a polypeptide that is at least 80 percent identical to the polypeptide LTWVKQAPGKGLKWMGW (SEQ ID NO: 13). In some embodiments, the anti-Tau phospho (Thr217) agent provided herein comprises a heavy chain fwr2 comprising a polypeptide that is at least 85 percent identical to the polypeptide LTWVKQAPGKGLKWMGW (SEQ ID NO: 13). In some embodiments, the anti-Tau phospho (Thr217) agent provided herein comprises a heavy chain fwr2 comprising a polypeptide that is at least 90 percent identical to the polypeptide LTWVKQAPGKGLKWMGW (SEQ ID NO: 13). In some embodiments, the anti-Tau phospho (Thr217) agent provided herein comprises a heavy chain fwr2 comprising a polypeptide that is at least 95 percent identical to the polypeptide LTWVKQAPGKGLKWMGW (SEQ ID NO: 13). In some embodiments, the anti-Tau phospho (Thr217) agent provided herein comprises a heavy chain fwr2 comprising a polypeptide that is at least 100 percent identical to the polypeptide

(SEQ ID NO: 13)
LTWVKQAPGKGLKWMGW

In some embodiments, an anti-Tau phospho (Thr217) agent provided herein comprises a heavy chain framework region 3 (fwr3) comprising the polypeptide THADDFTGRFAFSLETSASTAYLQIDNLNNEDTATYFC (SEQ ID NO: 14), or a polypeptide that is at least 80 percent identical to the polypeptide THADDFTGRFAFSLETSASTAYLQIDNLNNEDTATYFC (SEQ ID NO: 14). In some embodiments, the anti-Tau phospho (Thr217) agent provided herein comprises a heavy chain fwr3 comprising a polypeptide that is at least 85 percent identical to the polypeptide THADDFTGRFAFSLETSASTAYLQIDNLNNEDTATYFC (SEQ ID NO: 14). In some embodiments, the anti-Tau phospho (Thr217) agent provided herein comprises a heavy chain fwr3 comprising a polypeptide that is at least 90 percent identical to the polypeptide THADDFTGRFAFSLETSASTAYLQIDNLNNEDTATYFC (SEQ ID NO: 14). In some embodiments, the anti-Tau phospho (Thr217) agent provided herein comprises a heavy chain fwr3 comprising a polypeptide that is at least 95 percent identical to the polypeptide THADDFTGRFAFSLETSASTAYLQIDNLNNEDTATYFC (SEQ ID NO: 14). In some embodiments, the anti-Tau phospho (Thr217) agent provided herein comprises a heavy chain fwr3 comprising a polypeptide that is at least 100 percent identical to the polypeptide

(SEQ ID NO: 14)
THADDFTGRFAFSLETSASTAYLQIDNLNNEDTATYFC.

In some embodiments, an anti-Tau phospho (Thr217) agent provided herein comprises a heavy chain framework region 4 (fwr4) comprising the polypeptide WGQGTSVTVSS (SEQ ID NO: 15), or a polypeptide that is at least 80 percent identical to the polypeptide WGQGTSVTVSS (SEQ ID NO: 15). In some embodiments, the anti-Tau phospho (Thr217) agent provided herein comprises a heavy chain fwr4 comprising a polypeptide that is at least 85 percent identical to the polypeptide WGQGTSVTVSS (SEQ ID NO: 15). In some embodiments, the anti-Tau phospho (Thr217) agent provided herein comprises a heavy chain fwr4 comprising a polypeptide that is at least 90 percent identical to the polypeptide WGQGTSVTVSS (SEQ ID NO: 15). In some embodiments, the anti-Tau phospho (Thr217) agent provided herein comprises a heavy chain fwr4 comprising a polypeptide that is at least 95 percent identical to the polypeptide WGQGTSVTVSS (SEQ ID NO: 15). In some embodiments, the anti-Tau phospho (Thr217) agent provided herein comprises a heavy chain fwr4 comprising a polypeptide that is at least 100 percent identical to the polypeptide

(SEQ ID NO: 15)
WGQGTSVTVSS.

In some embodiments, an anti-Tau phospho (Thr217) agent provided herein comprises a light chain framework region 1 (fwr1) comprising the polypeptide DIVFTQSPASLAVSLGQRATISCRAS (SEQ ID NO: 16), or a polypeptide that is at least 80 percent identical to the polypeptide DIVFTQSPASLAVSLGQRATISCRAS (SEQ ID NO: 16). In some embodiments, the anti-Tau phospho (Thr217) agent provided herein comprises a light chain fwr1 comprising a polypeptide that is at least 85 percent identical to the polypeptide DIVFTQSPASLAVSLGQRATISCRAS (SEQ ID NO: 16). In some embodiments, the anti-Tau phospho (Thr217) agent provided herein comprises a light chain fwr1 comprising a polypeptide that is at least 90 percent identical to the polypeptide DIVFTQSPASLAVSLGQRATISCRAS (SEQ ID NO: 16). In some embodiments, the anti-Tau phospho (Thr217) agent provided herein comprises a light chain fwr1 comprising a polypeptide that is at least 95 percent identical to the polypeptide DIVFTQSPASLAVSLGQRATISCRAS (SEQ ID NO: 16). In some embodiments, the anti-Tau phospho (Thr217) agent provided herein comprises a light chain fwr1 comprising a polypeptide that is at least 100 percent identical to the polypeptide

(SEQ ID NO: 16)
DIVFTQSPASLAVSLGQRATISCRAS.

In some embodiments, an anti-Tau phospho (Thr217) agent provided herein comprises a light chain framework region 2 (fwr2) comprising the polypeptide MQWFQQKPGQPPKLLIY (SEQ ID NO: 17), or a polypeptide that is at least 80 percent identical to the polypeptide MQWFQQKPGQPPKLLIY (SEQ ID NO: 17). In some embodiments, the anti-Tau phospho (Thr217) agent provided herein comprises a light chain fwr2 comprising a polypeptide that is at least 85 percent identical to the polypeptide MQWFQQKPGQPPKLLIY (SEQ ID NO: 17). In some embodiments, the anti-Tau phospho (Thr217) agent provided herein comprises a light chain fwr2 comprising a polypeptide that is at least 90 percent identical to the polypeptide MQWFQQKPGQPPKLLIY (SEQ ID NO: 17). In some embodiments, the anti-Tau phospho (Thr217) agent provided herein comprises a light chain fwr2 comprising a polypeptide that is at least 95 percent identical to the polypeptide MQWFQQKPGQPPKLLIY (SEQ ID NO: 17). In some embodiments, the anti-Tau phospho (Thr217) agent provided herein comprises a light chain fwr2 comprising a polypeptide that is at least 100 percent identical to the polypeptide

(SEQ ID NO: 17)
MQWFQQKPGQPPKLLIY.

In some embodiments, an anti-Tau phospho (Thr217) agent provided herein comprises a light chain framework region 3 (fwr3) comprising the polypeptide NVESGVPARFSGSGSGTDFSLNIHPVEEDDMAMYFC (SEQ ID NO: 18), or a polypeptide that is at least 80 percent identical to the polypeptide NVESGVPARFSGSGSGTDFSLNIHPVEEDDMAMYFC (SEQ ID NO: 18). In some embodiments, the anti-Tau phospho (Thr217) agent provided herein comprises a light chain fwr3 comprising a polypeptide that is at least 85 percent identical to the polypeptide NVESGVPARFSGSGSGTDFSLNIHPVEEDDMAMYFC (SEQ ID NO: 18). In some embodiments, the anti-Tau phospho (Thr217) agent provided herein comprises a light chain fwr3 comprising a polypeptide that is at least 90 percent identical to the polypeptide NVESGVPARFSGSGSGTDFSLNIHPVEEDDMAMYFC (SEQ ID NO: 18). In some embodiments, the anti-Tau phospho (Thr217) agent provided herein comprises a light chain fwr3 comprising a polypeptide that is at least 95 percent identical to the polypeptide NVESGVPARFSGSGSGTDFSLNIHPVEEDDMAMYFC (SEQ ID NO: 18). In some embodiments, the anti-Tau phospho (Thr217) agent provided herein comprises a light chain fwr3 comprising a polypeptide that is at least 100 percent identical to the polypeptide

(SEQ ID NO: 18)
NVESGVPARFSGSGSGTDFSLNIHPVEEDDMAMYFC.

In some embodiments, an anti-Tau phospho (Thr217) agent provided herein comprises a light chain framework region 4 (fwr4) comprising the polypeptide FGGGTKLEIK (SEQ ID NO: 19), or a polypeptide that is at least 80 percent identical to the polypeptide FGGGTKLEIK (SEQ ID NO: 19). In some embodiments, the anti-Tau phospho (Thr217) agent provided herein comprises a light chain fwr4 comprising a polypeptide that is at least 85 percent identical to the polypeptide FGGGTKLEIK (SEQ ID NO: 19). In some embodiments, the anti-Tau phospho (Thr217) agent provided herein comprises a light chain fwr4 comprising a polypeptide that is at least 90 percent identical to the polypeptide FGGGTKLEIK (SEQ ID NO: 19). In some embodiments, the anti-Tau phospho (Thr217) agent provided herein comprises a light chain fwr4 comprising a polypeptide that is at least 95 percent identical to the polypeptide FGGGTKLEIK (SEQ ID NO: 19). In some embodiments, the anti-Tau phospho (Thr217) agent provided herein comprises a light chain fwr4 comprising a polypeptide that is at least 100 percent identical to the polypeptide FGGGTKLEIK (SEQ ID NO: 19).

Percent amino acid sequence identity with respect to a reference VH, VL, and CDR polypeptide sequences herein generally refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

### Competitor Agents

Provided herein are anti-Tau phospho (Thr217) agents that competitively bind, or are capable of competitively binding, with one or more anti-Tau phospho (Thr217) agents described herein. In particular, provided herein are anti-Tau phospho (Thr217) agents that compete, or are capable of competing, with one or more anti-Tau phospho (Thr217) agents described herein for binding to Tau phospho (Thr217). Such agents that compete, or are capable of competing, with anti-Tau phospho (Thr217) agents described herein may be referred to as competitor agents. In certain instances, an agent (i.e., competitor agent) may be considered to compete for binding to Tau phospho (Thr217) when the competitor binds to the same general region of Tau phospho (Thr217) as an anti-Tau phospho (Thr217) agent described herein. In certain instances, an agent (i.e., competitor agent) may be considered to compete for binding to Tau phospho (Thr217) when the competitor binds to the exact same region of Tau phospho (Thr217) as an anti-Tau phospho (Thr217) agent described herein (e.g., exact same peptide (linear epitope) or exact same surface amino acids [conformational epitope]). In certain instances, an agent (i.e., competitor agent) may be considered capable of competing for binding to Tau phospho (Thr217) when the competitor binds to the same general region of Tau phospho (Thr217) as an anti-Tau phospho (Thr217) agent described herein under suitable assay conditions. In certain instances, an agent (i.e., competitor agent) may be considered capable of competing for binding to Tau phospho (Thr217) when the competitor binds to the exact same region of Tau phospho (Thr217) as an anti-Tau phospho (Thr217) agent described herein (e.g., exact same peptide [linear epitope] or exact same surface amino acids [conformational epitope]) under suitable assay conditions.

In certain instances, an agent (i.e., competitor agent) may be considered to compete for binding to Tau phospho (Thr217) when the competitor blocks the binding of one or more anti-Tau phospho (Thr217) agents described herein to Tau phospho (Thr217). In certain instances, an agent (i.e., competitor agent) may be considered capable of competing for binding to Tau phospho (Thr217) when the competitor blocks the binding of one or more anti-Tau phospho (Thr217) agents described herein to Tau phospho (Thr217) under suitable assay conditions. Whether a competitor blocks the binding of one or more anti-Tau phospho (Thr217) agents described herein to Tau phospho (Thr217) may be determined using a suitable competition assay or blocking assay, such as, for example, a blocking assay as described in Example 4 herein. A competitor agent may block binding of one or more anti-Tau phospho (Thr217) agents described herein to Tau phospho (Thr217) in a competition or blocking assay by 50% or more, and conversely, one or more anti-Tau phospho (Thr217) agents described herein may block binding of the competitor agent to Tau phospho (Thr217) in a competition or blocking assay by about 50% or more. For example, an agent (i.e., competitor agent) may block binding of one or more anti-Tau phospho (Thr217) agents described herein to Tau phospho (Thr217) in a competition or blocking assay by about 55%, 60%, 65%, 70%, 75%, 80%, 85%, 90%, 95%, or 100%, and conversely, one or more anti-Tau phospho (Thr217) agents described herein may block binding of the competitor agent to Tau phospho (Thr217) in a competition or blocking assay by about 55%, 60%, 65%, 70%, 75%, 80%, 85%, 90%, 95%, or 100%.

In certain instances, an agent (i.e., competitor agent) may be considered to compete for binding to Tau phospho (Thr217) when the competitor binds to Tau phospho (Thr217) with a similar affinity as one or more anti-Tau phospho (Thr217) agents described herein. In certain instances, an agent (i.e., competitor agent) may be considered capable of competing for binding to Tau phospho (Thr217) when the competitor binds to Tau phospho (Thr217) with a similar affinity as one or more anti-Tau phospho (Thr217) agents described herein under suitable assay conditions. In some embodiments, an agent (i.e., competitor agent) is considered to compete for binding to Tau phospho (Thr217) when the competitor binds to Tau phospho (Thr217) with an affinity that is at least about 50% of the affinity of one or more anti-Tau phospho (Thr217) agents described herein. For example, an agent (i.e., competitor agent) may be considered to compete for binding to Tau phospho (Thr217) when the competitor binds to Tau phospho (Thr217) with an affinity that is at least about 55%, 60%, 65%, 70%, 75%, 80%, 85%, 90%, 95%, or 100% of the affinity of one or more anti-Tau phospho (Thr217) agents described herein. A competitor agent may comprise any feature described herein for anti-Tau phospho (Thr217) agents.

Also provided herein are anti-Tau phospho (Thr217) agents that bind to, or are capable of binding to, the same epitope as one or more anti-Tau phospho (Thr217) agents described herein. In particular, provided herein are anti-Tau phospho (Thr217) agents that compete with one or more anti-Tau phospho (Thr217) agents described herein for binding to the same epitope on Tau phospho (Thr217). Such agents that bind the same epitope may be referred to as epitope competitors. In certain instances, an epitope competitor may bind to the exact same region of Tau phospho (Thr217) as an anti-Tau phospho (Thr217) agent described herein (e.g., exact same peptide (linear epitope) or exact same surface amino acids [conformational epitope]). In certain instances, an epitope competitor blocks the binding of one or more anti-Tau phospho (Thr217) agents described herein to Tau phospho (Thr217). An epitope competitor may block binding of one or more anti-Tau phospho (Thr217) agents described herein to Tau phospho (Thr217) in a competition assay by about 50% or more, and conversely, one or more anti-Tau phospho (Thr217) agents described herein may block binding of the epitope competitor to Tau phospho (Thr217) in a competition assay by 50% or more. In certain instances, an epitope competitor binds to Tau phospho (Thr217) with a similar affinity as one or more anti-Tau phospho (Thr217) agents described herein. In some embodiments, an epitope competitor binds to Tau phospho (Thr217) with an affinity that is at least about 50% of the affinity of one or more anti-Tau phospho (Thr217) agents described herein. For example, an epitope competitor may bind to Tau phospho (Thr217) with an affinity that is at least about 55%, 60%, 65%, 70%, 75%, 80%, 85%, 90%, 95%, or 100% of the affinity of one or more anti-Tau phospho (Thr217) agents described herein. An epitope competitor may comprise any feature described herein for anti-Tau phospho (Thr217) agents.

### Antibodies

In some embodiments, an anti-Tau phospho (Thr217) agent is a Tau phospho (Thr217) binding antibody or antigen binding fragment thereof. An antibody as described herein generally includes of one or more polypeptides substantially encoded by immunoglobulin genes or fragments of immunoglobulin genes. Immunoglobulin genes generally include kappa, lambda, alpha, gamma, delta, epsilon, and mu constant region genes, as well as immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An antibody herein may be classified as IgG (e.g., IgG1, IgG2, IgG3, IgG4), IgM, IgA, IgD, or IgE.

An antibody may be monoclonal. A monoclonal antibody generally refers to an antibody obtained from a population of substantially homogeneous antibodies, i.e., the individual antibodies comprising the population are identical (as assessed at the level of Ig heavy and/or light chain amino acid sequence) and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier monoclonal indicates the character of the antibody as being obtained from a substantially homogeneous population of antibodies, and is not to be construed as requiring production of the antibody by any particular method. For example, the monoclonal antibodies to be used in accordance with the present technology may be made by a variety of techniques, including, but not limited to, hybridoma methods, recombinant DNA methods, phage-display methods, and methods utilizing transgenic animals containing all or part of the human immunoglobulin loci.

An antibody structural unit may be in the form of a tetramer. Each tetramer is composed of two identical pairs of polypeptide chains, each pair having one light chain (about 25 kD) and one heavy chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies typically include one or more variable regions or variable domains, which refer to portions of light and heavy chains of an antibody that include amino acid sequences of complementarity determining regions (CDRs, e.g., CDRH1, CDRH2, CDRH3, CDRL1, CDRL2, and CDRL3) and framework regions (FRs). The variable domain for heavy chains and light chains is commonly designated VH and VL, respectively. The variable domain is included on Fab, F(ab′)2, Fv and scFv antigen binding fragments, for example, and is involved in specific antigen recognition. A complementarity determining region (CDR) refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. The sequences of the framework regions of different light or heavy chains are relatively conserved within a species. The framework region of an antibody, which is the combined framework regions of the constituent light and heavy chains, serves to position and align the CDRs in three-dimensional space.

Antibodies typically have a heavy chain variable domain comprising an amino acid sequence represented by the formula: FRH1-CDRH1-FRH2-CDRH2-FRH3-CDRH3-FRH4, where FRH1-4 represent the four heavy chain framework regions and CDRH1-3 represent the three hypervariable regions of an antibody variable heavy domain. In some embodiments, FRH1-4 are derived from a consensus sequence (for example the most common amino acids of a class, subclass or subgroup of heavy or light chains of human immunoglobulins) or are derived from an individual human antibody framework region or from a combination of different framework region sequences.

Antibodies also typically have a light chain variable domain comprising an amino acid sequence represented by the formula: FRL1-CDRL1-FRL2-CDRL2-FRL3-CDRL3-FRL4, where FRL1-4 represent the four framework regions and CDRL1-3 represent the three hypervariable regions of an antibody variable light domain. In some embodiments, FRL1-4 are derived from a consensus sequence (for example the most common amino acids of a class, subclass or subgroup of heavy or light chains of human immunoglobulins) or are derived from an individual human antibody framework region or from a combination of different framework region sequences.

In some embodiments, an antibody is monovalent (an antibody with one antigen binding site). In some embodiments, an antibody is bivalent (an antibody with two antigen binding sites). In some embodiments, an antibody is in a multivalent form, e.g., a trivalent (an antibody with three antigen binding sites) or tetravalent form (an antibody with four antigen binding sites). In some embodiments, an antibody is monospecific (binds to one antigen or epitope). In some embodiments, an antibody is multi-specific (binds to more than one antigen or epitope; e.g., bispecific, trispecific, and the like).

Anti-Tau phospho (Thr217) antibodies herein may include VH-VL dimers, including single chain antibodies (antibodies that exist as a single polypeptide chain), such as single chain Fv antibodies (sFv or scFv) in which a variable heavy and a variable light domain are joined together (directly or through a peptide linker) to form a continuous polypeptide. A single chain Fv antibody typically is a covalently linked VH-VL which may be expressed from a nucleic acid including VH- and VL-encoding sequences either joined directly or joined by a peptide-encoding linker. While the VH and VL are connected to each other as a single polypeptide chain, the VH and VL domains typically associate non-covalently. VH and VL domains together typically include six complementarity determining regions (CDR) (three in each from the heavy and light chain) that contribute amino acid residues for antigen binding and confer antigen binding specificity to the antibody. In certain instances, a single variable domain (or half of an Fv comprising only three CDRs specific for an antigen) has the ability to recognize and bind antigen. A dsFv is an Fv with an engineered intermolecular disulfide bond, which stabilizes the VH-VL pair.

Anti-Tau phospho (Thr217) antibodies herein may include antigen binding fragments produced by the modification of whole antibodies. For example, pepsin digests an antibody C-terminal to the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region. While various antigen binding fragments are defined in terms of the digestion of an intact antibody, antigen binding fragments may be synthesized de novo either chemically or by utilizing recombinant DNA methodology. Thus, an antibody may refer to antigen binding fragments either produced by the modification of whole antibodies or synthesized using recombinant DNA methodologies.

Anti-Tau phospho (Thr217) antibodies herein may include antibody fragments or antigen binding fragments thereof that retain binding specificity. An antibody fragment or antigen binging fragment thereof may include a portion of an intact antibody (e.g., an antigen binding region of the intact antibody and/or a variable region of the intact antibody). Antibody fragments or antigen binding fragments thereof include but are not limited to Fv fragments, disulfide-linked Fvs (dsFv), Fab fragments, Fab′ fragments, and F(ab′) 2 fragments as described above. Antibody fragments or antigen binding fragments thereof also include but are not limited to Fd fragments (i.e., a fragment of an antibody containing a variable domain (VH) and one constant region domain (CHI) of an antibody heavy chain), Fd′ fragments (i.e., a fragment of an antibody containing one heavy chain portion of a F(ab′) 2 fragment), diabodies (i.e., dimeric scFvs), linear antibodies, and single-chain antibody molecules (e.g., single-chain Fvs [scFv] or single-chain Fabs [scFab]).

In some embodiments, an anti-Tau phospho (Thr217) antibody or antigen binding fragment thereof is a chimeric antibody. Chimeric antibodies may include immunoglobulin molecules in which the constant region, or a portion thereof, is altered, replaced, or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, and the like. Chimeric antibodies may also include immunoglobulin molecules in which the variable region, or a portion thereof, is altered, replaced, or exchanged with a variable region, or portion thereof, having a different or altered antigen specificity; or with corresponding sequences from another species or from another antibody class or subclass.

In some embodiments, an anti-Tau phospho (Thr217) antibody or antigen binding fragment thereof comprises one or more human framework regions. In some embodiments, an anti-Tau phospho (Thr217) antibody or antigen binding fragment thereof is humanized. Humanized antibodies generally refer to immunoglobulin molecules in which CDRs from a donor antibody (e.g., mouse, rabbit) are grafted onto human framework sequences. Humanized antibodies also may include residues of donor origin in the framework sequences. A humanized antibody also may include at least a portion of a human immunoglobulin constant region. Humanized antibodies also may include residues which are found neither in the recipient antibody nor in the imported CDR or framework sequences. Humanization can be performed using methods known in the art.

In some embodiments, an anti-Tau phospho (Thr217) antibody or antigen binding fragment thereof comprises one or more variations (e.g., amino acid substitutions, deletions, and/or insertions). An example method for identification of certain residues or regions of an antibody that are preferred locations for amino acid substitutions is alanine scanning mutagenesis. Here, a residue or group of target residues is identified (e.g., charged residues such as arg, asp, his, lys, and glu) and replaced by a neutral or negatively charged amino acid (most preferably alanine or polyalanine) to affect the interaction of the amino acids with an antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at a target codon or region, and the expressed antibody variants may be screened for the desired activity. Amino acid sequence insertions may include amino-terminal and/or carboxyl-terminal fusions ranging in length from one residue to polypeptides containing a hundred or more residues, as well as intra-sequence insertions of single or multiple amino acid residues. Examples of terminal insertions include an N-terminal methionyl residue or the antibody fused to an epitope tag. Other insertional variants include the fusion of an enzyme or a polypeptide that increases the serum half-life of the antibody to the N- or C-terminus of the antibody.

Sites for amino acid substitutions may include sites in the hypervariable regions and may include sites in the framework regions. Amino acid substitutions may include conservative substitutions or non-conservative substitutions. Examples of substitutions are listed below.
Ala (A): val; leu; ile
Arg (R): lys; gln; asn
Asn (N): gln; his; asp, lys; arg
Asp (D): glu; asn
Cys (C): ser; ala
Gin (Q): asn; glu
Glu (E): asp; gln
Gly (G): ala
His (H): asn; gln; lys; arg
Ile (I): leu; val; met; ala; phe; norleucine
Leu (L): norleucine; ile; val; met; ala; phe
Lys (K): arg; gln; asn
Met (M): leu; phe; ile
Phe (F): leu; val; ile; ala; tyr
Pro (P): ala
Ser(S): thr
Thr (T): ser
Trp (W): tyr; phe
Tyr (Y): trp; phe; thr; ser
Val (V): ile; leu; met; phe; ala; norleucine

Substantial modifications in the biological properties of an antibody may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues can be divided into groups based on common side-chain properties listed below.
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions generally entail exchanging a member of one of these classes for another class.

Any cysteine residue not involved in maintaining the proper conformation of an antibody also may be substituted, e.g., to improve the oxidative stability of the molecule and prevent aberrant crosslinking. Conversely, cysteine bond(s) may be added to the antibody to improve its stability (e.g., where the antibody is an antibody fragment such as an Fv fragment).

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity). In order to identify candidate hypervariable region sites for modification, alanine-scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution. Once such variants are generated, the panel of variants is subjected to screening and antibodies with superior properties in one or more relevant assays may be selected for further development.

Another type of amino acid variant of an antibody alters the original glycosylation pattern of the antibody. For example, one or more carbohydrate moieties found in the antibody may be deleted, and/or one or more glycosylation sites that are not present in the antibody may be added. Glycosylation of antibodies is typically either N-linked and/or or O-linked. N-linked refers to the attachment of a carbohydrate moiety to the side chain of an asparagine residue. The tripeptide sequences asparagine-X-serine and asparagine-X-threonine, where X is any amino acid except proline, are the most common recognition sequences for enzymatic attachment of a carbohydrate moiety to an asparagine side chain. Thus, the presence of either of these tripeptide sequences in a polypeptide creates a potential glycosylation site. O-linked glycosylation refers to the attachment of one of the sugars N-acetyl-galactosamine, galactose, or xylose to a hydroxyamino acid, most commonly serine or threonine, although 5-hydroxyproline or 5-hydroxylysine may also be used. Addition of glycosylation sites to an antibody may be accomplished by altering the amino acid sequence such that it contains one or more of the above-described tripeptide sequences (for N-linked glycosylation sites). The alteration may also be made by the addition of, or substitution by, one or more serine or threonine residues to the sequence of an original antibody (for O-linked glycosylation sites).

### Fc Polypeptide

In some embodiments, an antibody or antigen binding fragment thereof comprises a fragment crystallizable region (Fc region), also referred to as an Fc polypeptide. An Fc polypeptide is part of each of the two heavy chains in the antibody and can interact with certain cell surface receptors and certain components of the complement system. An Fc polypeptide typically includes the CH2 domain and the CH3 domain, which are immunoglobulin constant region domain polypeptides. In some embodiments, the Fc polypeptide in an antibody described herein can be a wild-type Fc polypeptide, e.g., a human IgG1 Fc polypeptide. In certain embodiments, an antibody described herein can comprise a wild-type Fc polypeptide having the following sequence:

(SEQ ID NO: 1)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

In come embodiments, an antibody or antigen binding fragment thereof comprises a variant of the wild-type Fc polypeptide that has at least 90% (e.g., 90%, 91%, 92%, 93%, 94%, 95%, 96%, 97%, 98%, 99%, or 99.5%) identity to the sequence of a wild-type Fc polypeptide (e.g., SEQ ID NO: 1) and at least one amino acid substitution relative to the sequence of a wild-type Fc polypeptide (e.g., SEQ ID NO: 1).

In some embodiments, an Fc polypeptide includes one or more modifications (e.g., one or more amino acid substitutions, insertions, or deletions relative to a comparable wild-type Fc region). Antibodies comprising modified Fc polypeptides typically have altered phenotypes relative to antibodies comprising wild-type Fc polypeptides. For example, antibodies comprising modified Fc polypeptides can have altered serum half-life, altered stability, altered susceptibility to cellular enzymes, and/or altered effector function (e.g., as assayed in an NK-dependent or macrophage-dependent assay).

In some embodiments, an Fc polypeptide in an antibody or antigen binding fragment thereof can include amino acid substitutions that modulate effector function. In certain embodiments, an Fc polypeptide in an antibody described herein can include amino acid substitutions that reduce or eliminate effector function. Illustrative Fc polypeptide amino acid substitutions that reduce effector function include, but are not limited to, substitutions in a CH2 domain, e.g., at positions 4 and 5 (position numbering relative to the sequence of SEQ ID NO: 1). For example, in some embodiments, one or both Fc polypeptides in an antibody described herein can comprise L4A and L5A substitutions.

Additional Fc polypeptide amino acid substitutions that modulate an effector function include, e.g., substitution at position 99 (position numbering relative to the sequence of SEQ ID NO: 1). For example, in some embodiments, one or both Fc polypeptides in an antibody described herein can comprise a P99G substitution. In certain embodiments, one or both Fc polypeptides in an antibody described herein can have L4A, L5A, and P99G substitutions.

In some embodiments, an Fc polypeptide includes one or more modifications that alter (relative to a wild-type Fc polypeptide) the Ratio of Affinities of the modified Fc polypeptide to an activating Fc^{γ}R (such as Fc^{γ}RIIA or Fc^{γ}RIIIA) relative to an inhibiting Fc^{γ}R (such as Fc^{γ}RIIB):

$$Ratio of Affinities = \frac{WT to variant change in affinity to {Fc}^{γ} {R}_{activating}}{WT to variant change in affinity to {Fc}^{γ} {R}_{inhibiting}}$$

Where a modified Fc polypeptide has a Ratio of Affinities greater than 1, an antibody herein may have particular use in providing a therapeutic or prophylactic treatment of a disease, disorder, or infection, or the amelioration of a symptom thereof, where an enhanced efficacy of effector cell function mediated by Fc^{γ}R is desired, e.g., cancer or infectious disease. Where a modified Fc region has a Ratio of Affinities less than 1, an antibody herein may have particular use in providing a therapeutic or prophylactic treatment of a disease or disorder, or the amelioration of a symptom thereof, where a decreased efficacy of effector cell function mediated by Fc^{γ}R is desired, e.g., autoimmune or inflammatory disorders. The following are examples of single, double, triple, quadruple, and quintuple amino acid substitutions in an Fc polypeptide that provide a Ratio of Affinities greater than 1 or less than 1.

TABLE 1
Example Fc amino acid substitutions

| Ratio | Single | Double | Triple | Quadruple | Quintuple |
|---|---|---|---|---|---|
| >1 | F243L | F243L, R292P | F243L, P247L, | L234F, F243L, | L235V, F243L, |
| | | | N421K | R292P, Y300L | R292P, Y300L, |
| | | | | | P396L |
| | D270E | F243L, Y300L | F243L, R292P, | L235I, F243L | L235P, F243L, |
| | | | Y300L | R292P, Y300L | R292P, Y300L, |
| | | | | | P396L |
| | R292G | F243L, P396L | F243L, R292P, | L235Q, F243L, | F243L, R292P, |
| | | | V305I | R292P, Y300L | V305I, Y300L, |
| | | | | | P396L |
| | R292P | D270E, P396L | F243L, R292P, | F243L, P247L, | |
| | | | P396L | D270E, N421K | |
| | | R292P, Y300L | F243L, Y300L, | F243L, R255L, | |
| | | | P396L | D270E, P396L | |
| | | R292P, V305I | P247L, D270E, | F243L, D270E, | |
| | | | N421K | G316D, R416G | |
| | | R292P, P396L | R255L, D270E, | F243L, D270E, | |
| | | | P396L | K392T, P396L | |
| | | Y300L, P396L | D270E, G316D, | F243L, D270E, | |
| | | | R416G | P396L, Q419H | |
| | | P396L, Q419H | D270E, K392T, | F243L, R292P, | |
| | | | P396L | Y300L, P396L | |
| | | | D270E, P396L, | F243L, R292P, | |
| | | | Q419H | V305I, P396L | |
| | | | V284M, R292L, | P247L, D270E, | |
| | | | K370N | Y300L, N421K | |
| | | | R292P, Y300L, | R255L, D270E, | |
| | | | P396L | R292G, P396L | |
| | | | | R255L, D270E, | |
| | | | | Y300L, P396L | |
| | | | | D270E, G316D, | |
| | | | | P396L, R416G | |
| <1 | Y300L | F243L, P396L | F243L, R292P, | | |
| | | | V305I | | |
| | P396L | P247L, N421K | | | |
| | | R255L, P396L | | | |
| | | R292P, V305I | | | |
| | | K392T, P396L | | | |
| | | P396L, Q419H | | | |

### Antigens

An antibody may bind to one or more antigens. An antigen generally refers to a molecule, compound, or complex that is recognized by an antibody, i.e., can be specifically bound by the antibody. The term antigen may be used interchangeably with immunogen, antibody target, target analyte, and the like. An antigen may refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). The term antigen does not necessarily indicate that the molecule is immunogenic in every context, but generally indicates that it can be targeted by an antibody.

Antibodies typically bind to an epitope on an antigen. An epitope is the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, at least 4, at least 5, at least 6, at least 7, at least 8, or at least 9 amino acids in a unique spatial conformation. In certain instances, an epitope includes 8-10 amino acids in a unique spatial conformation. Methods of determining spatial conformation of epitopes include, for example, x-ray crystallography and 2-dimensional nuclear magnetic resonance.

The terms specific for, specifically binds, and like terms generally refer to a molecule (e.g., antibody or antigen binding fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a target will typically bind the target with at least a 2-fold greater affinity than a non-target. Specificity can be determined using standard methods, e.g., solid-phase ELISA immunoassays. The term binds with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least about 2/3 of the antibody targets in a solution (e.g., at least about 75%, 80%, 85%, 90%, 91%, 92%, 93%, 94%, 95%, 96%, 97%, 98%, 99%, or 100% of the antibody targets in a solution).

### Modified Agents/Antibodies

In some embodiments, an agent/antibody comprises one or more modifications. For example, immunoconjugates comprising an antibody described herein may be conjugated to a cytotoxic agent such as a toxin (e.g., an enzymatically active toxin of bacterial, fungal, plant or animal origin, or fragments thereof), a radioactive isotope (for example, a radioconjugate), or a cytotoxic drug. Such conjugates are sometimes referred to as antibody-drug conjugates or ADCs. Conjugates can be made using any suitable bifunctional protein coupling agent such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In some embodiments, an agent/antibody disclosed herein may be formulated as an immunoliposome. Liposomes containing an antibody may be prepared by a suitable method known in the art. Liposomes with enhanced circulation time may be generated. For example, liposomes can be generated by a reverse phase evaporation method with a lipid composition comprising phosphatidylcholine, cholesterol, and PEG-derivatized phosphatidylethanolamine (PEG-PE). Liposomes may be extruded through filters of defined pore size to yield liposomes with a desired diameter. Fab′ fragments of an antibody provided herein may be conjugated to the liposomes via a disulfide interchange reaction, for example. Other active ingredients may be contained within the liposome.

In some embodiments, enzymes or other polypeptides may be covalently bound to an agent/antibody disclosed herein by a suitable technique such as the use of the heterobifunctional cross-linking reagents. In some embodiments, fusion proteins comprising at least the antigen binding region of an antibody provided herein linked to at least a functionally active portion of an enzyme can be constructed using recombinant DNA techniques.

In certain embodiments, it may be desirable to use an antigen binding fragment, rather than an intact antibody, to increase penetration of target tissues and cells, for example. In such instances, it may be desirable to modify the antigen binding fragment in order to increase its serum half-life. This may be achieved, for example, by incorporation of a salvage receptor binding epitope into the antigen binding fragment (e.g., by mutation of the appropriate region in the antigen binding fragment or by incorporating the epitope into a peptide tag that is then fused to the antigen binding fragment at either end or in the middle, e.g., by DNA or peptide synthesis.

In some embodiments, an antibody or antigen binding fragment thereof comprises, is conjugated to, or is hybridized to one or more oligonucleotides. In some embodiments, the oligonucleotide contains a barcode sequence (e.g., a sample barcode sequence). In some embodiments, the oligonucleotide further contains a binding site for a primer and/or an anchor. In some embodiments, a detectable marker or label (e.g., a radioisotope or fluorophore) is conjugated to the oligonucleotide. In some embodiments, the oligonucleotide is a polymeric sequence. The terms oligonucleotide and polynucleotide are used interchangeably to refer to a single-stranded multimer of nucleotides from about 2 to about 500 nucleotides in length. Oligonucleotides may be synthetic, made enzymatically (e.g., via polymerization), or using a “split-pool” method. Oligonucleotides may include ribonucleotide monomers (i.e., can be oligoribonucleotides) and/or deoxyribonucleotide monomers (i.e., oligodeoxyribonucleotides). Oligonucleotides may include a combination of both deoxyribonucleotide monomers and ribonucleotide monomers in the oligonucleotide (e.g., random or ordered combination of deoxyribonucleotide monomers and ribonucleotide monomers). Oligonucleotides may include one or more functional moieties that are attached (e.g., covalently or non-covalently) to another structure.

An anchor generally refers to a polymer, e.g., a polynucleotide or oligonucleotide, which is designed to hybridize to a complementary oligonucleotide sequence. In some embodiments, an anchor is designed for the purpose of generating a double stranded construct oligonucleotide sequence. In some embodiments, an anchor is positioned at the 3′ end of the construct oligonucleotide sequence. In some embodiments, an anchor is positioned at the 5′ end of a construct oligonucleotide sequence. Each anchor may be specific for its intended complementary sequence.

In some embodiments, a binding site for a primer is a functional component of the oligonucleotide which itself is an oligonucleotide or polynucleotide sequence that provides an annealing site for amplification of the oligonucleotide. A binding site for a primer can be formed of polymers of DNA, RNA, PNA, modified bases or combinations of these bases, or polyamides, etc. In some embodiments, a binding site for a primer is about 10 of such monomeric components, e.g., nucleotide bases, in length. In some embodiments, a binding site for a primer is at least about 5 to 100 monomeric components, e.g., nucleotides, in length. In certain embodiments, a binding site for a primer can be a generic sequence suitable as an annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (I), ramification amplification method, and RNA-seq.

In some embodiments, a modification is introduced into an antibody (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of polypeptides, for example, to prevent rapid filtration into the urine. In some embodiments, a lysine residue in a sequence is conjugated to PEG directly or through a linker. Such a linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art.

Covalent modifications of an antibody are also included within the scope of this technology. For example, modifications may be made by chemical synthesis or by enzymatic or chemical cleavage of an antibody. Other types of covalent modifications of an antibody are introduced into the molecule by reacting targeted amino acid residues of the antibody with an organic derivatizing agent that is capable of reacting with selected side chains or the N- or C-terminal residues. An example covalent modification of an antibody involves linking the antibody to one of a variety of non-proteinaceous polymers, e.g., polyethylene glycol, polypropylene glycol, or polyoxyalkylenes.

### Labels and Solid Supports

In some embodiments, an anti-Tau phospho (Thr217) agent/antibody comprises a detectable marker or label. In some embodiments, an anti-Tau phospho (Thr217) agent/antibody is conjugated to a detectable marker or label.

For example, for research and diagnostic applications, an anti-Tau phospho (Thr217) agent/antibody may be labeled with a detectable moiety. Any suitable marker, label, or moiety may be associated with or conjugated to an anti-Tau phospho (Thr217) agent/antibody herein. In some embodiments, an anti-Tau phospho (Thr217) agent/antibody is labeled with one or more radioisotopes such as, for example, ³⁵S, ¹⁴C, ¹²⁵I, ³H, and ¹³¹I. The antibody can be labeled with the radioisotope using techniques known in the art, and radioactivity can be measured using scintillation counting, for example. In some embodiments, an anti-Tau phospho (Thr217) agent/antibody is labeled with one or more fluorescent labels such as, for example, rare earth chelates (europium chelates), fluorescein and its derivatives, rhodamine and its derivatives, dansyl, Lissamine, phycoerythrin, Texas Red and Brilliant Violet™. Fluorescent labels may be conjugated to an antibody using methods known in the art. Fluorescence can be quantified using a flow cytometer, imaging microscope, or fluorimeter, for example.

In some embodiments, an anti-Tau phospho (Thr217) agent/antibody is labeled with one or more enzyme-substrate labels. An enzyme can catalyze a chemical alteration of a chromogenic substrate that can be measured using various techniques. For example, an enzyme may catalyze a color change in a substrate, which can be measured spectrophotometrically. Alternatively, an enzyme may alter the fluorescence or chemiluminescence of a substrate. Techniques for quantifying a change in fluorescence are known in the art. For example, a chemiluminescent substrate becomes electronically excited by a chemical reaction and may then emit light that can be measured (using a chemilluminometer, for example) or donates energy to a fluorescent acceptor. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies known in the art.

In certain instances, a label is indirectly conjugated with the agent/antibody. For example, an antibody can be conjugated with biotin and any of the labels described above can be conjugated with avidin, or vice versa. Biotin binds selectively to avidin and thus, the label can be conjugated with the antibody in this indirect manner. Alternatively, to achieve indirect conjugation of a label with an antibody, the antibody may be conjugated with a small hapten (e.g., digoxin) and a label described above can be conjugated with an anti-hapten antibody (e.g., anti-digoxin antibody). Thus, indirect conjugation of a label with an antibody can be achieved.

In some embodiments, an anti-Tau phospho (Thr217) agent/antibody need not be labeled, and the presence thereof can be detected, e.g., using a labeled antibody which binds to an anti-Tau phospho (Thr217) antibody.

In some embodiments, an anti-Tau phospho (Thr217) agent/antibody herein is immobilized on a solid support or substrate. In some embodiments, an anti-Tau phospho (Thr217) agent/antibody herein is non-diffusively immobilized on a solid support (e.g., the anti-Tau phospho (Thr217) agent/antibody does not detach from the solid support). A solid support or substrate can be any physically separable solid to which an anti-Tau phospho (Thr217) agent/antibody can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass [CPG]), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe²⁺ and Fe³⁺). An anti-Tau phospho (Thr217) agent/antibody may be linked to a solid support by covalent bonds or by non-covalent interactions and may be linked to a solid support directly or indirectly (e.g., via an intermediary agent such as a spacer molecule or biotin).

### Nucleic Acids, Vectors, Host Cells, and Recombinant Methods

Provided herein are isolated nucleic acids encoding an anti-Tau phospho (Thr217) agent (e.g., anti-Tau phospho (Thr217) antibody), vectors and host cells comprising the nucleic acid, and recombinant techniques for the production of the agent or antibody.

Provided herein are nucleic acids (e.g., isolated nucleic acids) comprising a nucleotide sequence that encodes an anti-Tau phospho (Thr217) agent or antibody, or fragment thereof. In some embodiments, a nucleic acid encodes an immunoglobulin heavy chain variable domain of an anti-Tau phospho (Thr217) agent provided herein. In some embodiments, a nucleic acid encodes an immunoglobulin light chain variable domain of an anti-Tau phospho (Thr217) agent provided herein. In some embodiments, a nucleic acid encodes an immunoglobulin heavy chain variable domain and an immunoglobulin light chain variable domain of an anti-Tau phospho (Thr217) agent provided herein. In some embodiments, a nucleic acid comprises a nucleotide sequence that encodes an amino acid sequence of any one of SEQ ID NOs. 2-9. For example, a nucleic acid may comprise a nucleotide sequence that encodes a CDR amino acid sequence of any one of SEQ ID NOs. 4-9. A nucleic acid may comprise a nucleotide sequence that encodes an immunoglobulin heavy chain variable domain amino acid sequence of SEQ ID NO: 2. A nucleic acid may comprise a nucleotide sequence that encodes an immunoglobulin light chain variable domain amino acid sequence of SEQ ID NO: 3.

For recombinant production of an anti-Tau phospho (Thr217) agent or antibody, a nucleic acid encoding the anti-Tau phospho (Thr217) agent or antibody may be isolated and inserted into a replicable vector for further cloning and/or expression. In certain instances, an anti-Tau phospho (Thr217) agent or antibody may be produced by homologous recombination. DNA encoding an anti-Tau phospho (Thr217) agent or antibody can be readily isolated and sequenced using conventional procedures (e.g., by using oligonucleotide probes that are capable of binding specifically to genes encoding the heavy and light chains of the antibody). Any suitable vector may be used. Vector components generally include, but are not limited to, one or more of a signal sequence, an origin of replication, one or more marker genes, an enhancer element, a promoter, and a transcription termination sequence.

Suitable host cells for cloning and/or expressing DNA in vectors include prokaryote cells, yeast cells, and higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. In certain instances, eukaryotic microbes such as filamentous fungi or yeast are suitable cloning and/or expression hosts for anti-Tau phospho (Thr217) agent/antibody-encoding vectors. Saccharomyces cerevisiae, or common baker's yeast, is commonly used among lower eukaryotic host microorganisms. A number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24, 178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for expression of anti-Tau phospho (Thr217) agents/antibodies may be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silk moth) have been identified. A variety of viral strains for transfection are publicly available, e.g., the L-1 variant of Autographa californica NPV and the Bm-5 strain of Bombyx mori NPV. Plant cell cultures of cotton, corn, potato, soybean, petunia, tomato, and tobacco can also be utilized as hosts.

Suitable host cells for expression of anti-Tau phospho (Thr217) agents/antibodies also may include vertebrate cells (e.g., mammalian cells). Vertebrate cells may be propagated in culture (tissue culture). Examples of useful mammalian host cell lines include monkey kidney CV1 line transformed by SV40 (COS-7, ATCC CRL 1651); human embryonic kidney line (293, ATCC CRL-1573 or 293 cells subcloned for growth in suspension culture); baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells may be transformed with the above-described expression or cloning vectors for antibody production and cultured in conventional nutrient media modified as appropriate for inducing promoters, selecting transformants, and/or amplifying the genes encoding the desired sequences. Host cells used to produce an agent/antibody herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. Certain culture conditions, such as temperature, pH, and the like, may include conditions previously used with the host cell selected for expression.

When using recombinant techniques, an agent/antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. The agent/antibody composition prepared from the cells can be purified using, for example, hydroxylapatite chromatography, gel electrophoresis, dialysis, and affinity chromatography, with affinity chromatography being the preferred purification technique. Protein A can be used to purify antibodies that are based on human heavy chains. The suitability of protein A as an affinity ligand depends on the species and isotype of any immunoglobulin Fc domain that is present in the antibody. Protein G may be for mouse isotypes and for human g3. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

### Pharmaceutical Formulations, Dosing, and Routes of Administration

Provided herein are therapeutic compositions comprising an anti-Tau phospho (Thr217) agent/antibody described herein, and a pharmaceutically acceptable excipient. In some embodiments, an anti-Tau phospho (Thr217) antibody or antigen binding fragment thereof may be formulated in a pharmaceutical composition that is useful for a variety of purposes, including the treatment of diseases or disorders (e.g., neurological diseases or disorders, neurodegenerative diseases or disorders, tauopathies, Alzheimer's disease, Pick's disease, frontotemporal dementia, corticobasal degeneration, argyrophilic grain disease, progressive supranuclear palsy, Huntington's disease, Parkinson's disease). Pharmaceutical compositions comprising one or more antibodies may be administered using a pharmaceutical device to a patient in need thereof, and according to one embodiment of the technology, kits are provided that include such devices. Such devices and kits may be designed for routine administration, including self-administration, of the pharmaceutical compositions herein.

Provided herein are therapeutic compositions comprising an anti-Tau phospho (Thr217) agent/antibody described herein and a pharmaceutically acceptable carrier, excipient, or stabilizer. Therapeutic formulations of an anti-Tau phospho (Thr217) antibody may be prepared for storage by mixing the agent or antibody having the desired degree of purity with physiologically and/or pharmaceutically acceptable carriers, excipients, or stabilizers, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

Formulations herein may also contain more than one active compound as necessary for the particular indication being treated, preferably those with complementary activities that do not adversely affect each other. Such molecules are suitably present in combination in amounts that are effective for the purpose intended. Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the agent/antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides, copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods.

For therapeutic applications, antibodies provided herein may be administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, or inhalation routes. For the prevention or treatment of disease, the appropriate dosage of agent or antibody will depend on the type of disease to be treated, the severity and course of the disease, whether the antibody is administered for preventative or therapeutic purposes, previous therapy, the patient's clinical history and response to the antibody, and the discretion of the health professional. The antibody may be suitably administered to the patient at one time or over a series of treatments.

Depending on the type and severity of the disease, about 1 μg/kg to about 50 mg/kg (e.g., 0.1-20 mg/kg) of antibody may be an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment may be repeated until a desired suppression of disease symptoms occurs. However, other dosage regimens may be useful. The progress of this therapy is easily monitored by conventional techniques and assays, including, for example, radiographic imaging. Detection methods using the antibody to determine Tau phospho (Thr217) levels in bodily fluids or tissues may be used in order to optimize patient exposure to the therapeutic antibody.

In some embodiments, a composition comprising an antibody herein can be administered as a monotherapy, and in some embodiments, the composition comprising the antibody can be administered as part of a combination therapy. Accordingly, provided herein are therapeutic compositions where the anti-Tau phospho (Thr217) agent/antibody is used as an adjuvant or in conjunction with an adjuvant. In some cases, the effectiveness of the antibody in preventing or treating diseases may be improved by administering the antibody serially or in combination with another drug that is effective for those purposes, such as a chemotherapeutic drug for treatment of cancer or a microbial infection. In other cases, the antibody may serve to enhance or sensitize cells to chemotherapeutic treatment, thus permitting efficacy at lower doses and with lower toxicity.

Certain combination therapies include, in addition to administration of the composition comprising an antibody that blocks Tau phospho (Thr217) activity, delivering a second therapeutic regimen selected from the group consisting of a chemotherapeutic agent, radiation therapy, surgery, and a combination of any of the foregoing. Such other agents may be present in the composition being administered or may be administered separately. Also, the antibody may be suitably administered serially or in combination with the other agent or modality, e.g., chemotherapeutic drug or radiation for treatment of cancer, infection, and the like, or an immunosuppressive drug.

### Research and Diagnostic

Provided herein are diagnostic reagents comprising anti-Tau phospho (Thr217) agent/antibody described herein. For example, antibodies provided herein may be used to detect and/or purify Tau phospho (Thr217) from bodily fluid(s) or tissues. Anti-Tau phospho (Thr217) antibodies, for example, may be useful in diagnostic assays for Tau phospho (Thr217), e.g., detecting its presence in specific cells (e.g., neurons, astrocytes, oligodendrocytes, glial cells), tissues (brain tissue, CNS tissue), or bodily fluids (blood, plasma, serum, cerebrospinal fluid). Such diagnostic methods may be useful in diagnosis of, e.g., neurological diseases or disorders, neurodegenerative diseases or disorders, tauopathies, Alzheimer's disease, Pick's disease, frontotemporal dementia, corticobasal degeneration, argyrophilic grain disease, progressive supranuclear palsy, Huntington's disease, Parkinson's disease. Also provided herein are methods for detecting Tau phospho (Thr217) and/or measuring Tau phospho (Thr217) levels in a subject or in a sample from a subject. For example, a method may comprise contacting a sample (e.g., a biological sample known or suspected to contain Tau phospho (Thr217)) with an antibody provided herein, and, if the sample contains Tau phospho (Thr217), detecting Tau phospho (Thr217): antibody complexes. In some embodiments, a Tau phospho (Thr217) detection method is performed in vitro. In some embodiments, a Tau phospho (Thr217) detection method is performed in vivo. For in vivo diagnostic assays, the antibody may be labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography.

Also provided herein are reagents comprising anti-Tau phospho (Thr217) agent/antibody described herein for non-diagnostic use. Also provided herein are reagents comprising anti-Tau phospho (Thr217) agent/antibody described herein for non-therapeutic use. Also provided herein are reagents comprising anti-Tau phospho (Thr217) agent/antibody described herein for non-diagnostic and non-therapeutic use. For example, provided herein are reagents comprising an anti-Tau phospho (Thr217) agent/antibody described herein for use in research applications. Research applications may include investigating Tau phospho (Thr217) and its role in the development of one or more of neurological diseases or disorders, neurodegenerative diseases or disorders, tauopathies, Alzheimer's disease, Pick's disease, frontotemporal dementia, corticobasal degeneration, argyrophilic grain disease, progressive supranuclear palsy, Huntington's disease, and Parkinson's disease. In certain instances, research applications may include investigating Tau phospho (Thr217) and its role in microtubule assembly, microtubule stabilization, formation of paired helical filaments, or formation of neurofibrillary tangles. In certain instances, research applications may include investigating conformation changes in the structure of Tau.

Also provided herein are methods for detecting Tau phospho (Thr217) and/or measuring Tau phospho (Thr217) levels in a non-biological sample. For example, a method may comprise contacting a non-biological sample (e.g., a laboratory research sample known or suspected to contain Tau phospho (Thr217)) with an antibody provided herein, and, if the sample contains Tau phospho (Thr217), detecting Tau phospho (Thr217): antibody complexes. Laboratory research samples may include non-human animal models, samples from non-human animal models, cell lines, products produced by cell lines, and the like.

Anti-Tau phospho (Thr217) agents and antibodies provided herein may be employed in any suitable detection assay, such as flow cytometry, immunohistochemistry, immunofluorescence, mass cytometry, competitive binding assays, direct and indirect sandwich assays, and immunoprecipitation assays.

Flow cytometry and mass cytometry assays generally involve the use of a single primary antibody to specifically identify the presence of the target molecule expressed on the surface of a dispersed suspension of individual cells. The dispersed cells are typically obtained from a biological fluid sample, e.g., blood, but may also be obtained from a dispersion of single cells prepared from a solid tissue sample such as a tumor biopsy. The primary antibody may be directly conjugated with a detectable moiety, e.g., a fluorophore such as phycoerythrin for flow cytometry or a heavy metal chelate for mass cytometry. Alternatively, the primary antibody may be unlabeled or labeled with an undetectable tag such as biotin, and the primary antibody is then detected by a detectably labeled secondary antibody that specifically recognizes the primary antibody itself or the tag on the primary antibody. The labeled cells are then analyzed in an instrument capable of single cell detection, e.g., flow cytometer, mass cytometer, fluorescence microscope or brightfield light microscope, to identify those individual cells in the dispersed population or tissue sample that express the target recognized by the primary antibody. In certain instances, fixed and permeabilized cells may be used, and in such instances, intracellular Tau phospho (Thr217) may be detected.

Sandwich assays involve the use of two antibodies, each capable of binding to a different immunogenic portion, or epitope, of the protein that is detected. In a sandwich assay, the test sample analyte is bound by a first antibody that is immobilized on a solid support, and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. In a cell ELISA, the target cell population may be attached to the solid support using antibodies first attached to the support and that recognize different cell surface proteins. These first antibodies capture the cells to the support. In certain instances, fixed and permeabilized cells may be used, and in such instances, intracellular Tau phospho (Thr217) may be detected.

In some embodiments, an agent/antibody provided herein is formulated for immunohistochemical analysis. In some embodiments, immunohistochemical analysis includes the use of samples. In some embodiments, immunohistochemical analysis includes the use of blood and/or tissue samples. A sample may be fresh or frozen or may be embedded in paraffin and fixed with a preservative such as formalin. In some embodiments, a sample is a formalin-fixed paraffin-embedded (FFPE) sample. In some embodiments, an FFPE sample is saturated with formalin (i.e. formaldehyde) and then embedded in a block of paraffin wax. In some embodiments, an FFPE sample is stable at room temperature. In some embodiments, all or most of the structures in an FFPE sample are preserved. In some embodiments, the intracellular and surface proteins in an FFPE sample are preserved. In some embodiments, mRNA in an FFPE sample is preserved. In some embodiments, mRNA, intracellular proteins, and surface proteins in an FFPE sample are preserved. In some embodiments, surface proteins in an FFPE sample are denatured.

In some embodiments, an anti-Tau phospho (Thr217) antibody or antigen binding fragment thereof provided herein is capable of detecting Tau phospho (Thr217) in an FFPE sample. In some embodiments, an anti-Tau phospho (Thr217) antibody or antigen binding fragment thereof provided herein is capable of detecting intracellular Tau phospho (Thr217) in an FFPE sample. In some embodiments, an anti-Tau phospho (Thr217) antibody or antigen binding fragment thereof provided herein is capable of detecting Tau phospho (Thr217) in an FFPE sample. In some embodiments, an anti-Tau phospho (Thr217) antibody or antigen binding fragment thereof provided herein is capable of detecting intracellular Tau phospho (Thr217) and Tau phospho (Thr217) in an FFPE sample.

In some embodiments, a sample is a fresh sample that has been frozen. In some embodiments, a sample is a fresh sample that has been cryogenically frozen. In some embodiments, a sample is flash frozen. In some embodiments, a sample is flash frozen and stored at −80° C. In some embodiments, all or most of the structures in a flash frozen sample are preserved. In some embodiments, intracellular and surface proteins in a flash frozen sample are preserved. In some embodiments, mRNA in a flash frozen sample is preserved. In some embodiments, mRNA, intracellular proteins, and surface proteins in a flash frozen sample are preserved. In some embodiments, surface proteins in a flash frozen sample are denatured.

In some embodiments, an anti-Tau phospho (Thr217) antibody or antigen binding fragment thereof provided herein is capable of detecting Tau phospho (Thr217) in a frozen sample. In some embodiments, an anti-Tau phospho (Thr217) antibody or antigen binding fragment thereof provided herein is capable of detecting intracellular Tau phospho (Thr217) in a frozen sample. In some embodiments, an anti-Tau phospho (Thr217) antibody or antigen binding fragment thereof provided herein is capable of detecting Tau phospho (Thr217) on the surface of a frozen sample. In some embodiments, an anti-Tau phospho (Thr217) antibody or antigen binding fragment thereof provided herein is capable of detecting intracellular Tau phospho (Thr217) and Tau phospho (Thr217) in a frozen sample.

### Detection of Tau Phospho (Thr217)

Provided herein are antibodies and methods for detecting Tau phospho (Thr217). In some embodiments, antibodies and methods are provided for detecting Tau phospho (Thr217) in a biological sample. In some embodiments, Tau phospho (Thr217) is detected intracellularly. In some embodiments, Tau phospho (Thr217) is detected intercellularly. In some embodiments, Tau phospho (Thr217) is detected within a cell. In some embodiments, detection of Tau phospho (Thr217) is in vitro. In some embodiments, detection of Tau phospho (Thr217) is in vivo. In some embodiments, a biological sample is a solid tissue, fluid, or cell. Solid tissue samples may comprise solid tissue from one or more of adipose tissue, bladder, bone, brain, breast, cervix, endothelium, gallbladder, kidney, liver, lung, lymph, ovary, prostate, salivary gland, stomach, testis, thyroid, urethra, uterus, vagina, and vulva. Fluid samples may comprise one or more of amniotic fluid, bile, blood, breast milk, breast fluid, cerebrospinal fluid, lavage fluid, lymphatic fluid, mucous, plasma, saliva, semen, serum, spinal fluid, sputum, tears, umbilical cord blood, urine, and vaginal fluid.

In some embodiments, a sample comprises CNS cells. In some embodiments, a sample comprises one or more CNS cells chosen from neurons, astrocytes, oligodendrocytes, and glial cells. In some embodiments, a sample comprises non-CNS cells. For example, a sample may comprise one or more of fibroblasts, vascular smooth muscle cells, and endothelial cells. In some embodiments, a sample comprises diseased cells.

In some embodiments, the biological sample is from a healthy subject. In some embodiments, the sample is from a subject with a disease or condition. In some embodiments, the detection of Tau phospho (Thr217) indicates the presence or absence of a disease or disorder. In some embodiments, the disease or disorder is a neurologic disease or disorder, a neurodegenerative disease or disorder, a tauopathy, a cancer, an autoimmune disorder, an inflammatory disorder, or an infection. In some embodiments, the disease or disorder is Alzheimer's disease, Pick's disease, frontotemporal dementia, corticobasal degeneration, argyrophilic grain disease, progressive supranuclear palsy, Huntington's disease, or Parkinson's disease. In some embodiments, the disease or disorder is associated with Tau phospho (Thr217) expression. In some embodiments, the disease or disorder is associated with aberrant Tau phospho (Thr217) expression. In some embodiments, the disease or disorder is associated with the CNS. In some embodiments, the disease or disorder is associated with neurons. In some embodiments, the disease or disorder is associated with astrocytes. In some embodiments, the disease or disorder is associated with glial cells. In some embodiments, the disease or disorder is associated with oligodendrocytes.

### Kits

Provided herein are kits comprising an anti-Tau phospho (Thr217) agent/antibody described herein. A kit generally refers to a packaged combination of reagents in predetermined amounts with instructions for use (e.g., instructions for performing a diagnostic assay). In some embodiments, the kit is a diagnostic kit configured to detect Tau phospho (Thr217) in a sample (e.g., a biological sample). In some embodiments, the kit is a non-diagnostic kit configured to detect Tau phospho (Thr217) in biological and non-biological samples only for research purposes (e.g., a research sample from laboratory animals). Where the anti-Tau phospho (Thr217) agent is labeled with a fluorophore, the kit may include an identical isotype negative control irrelevant antibody to control for non-specific binding of the anti-Tau phospho (Thr217) agent. Where the anti-Tau phospho (Thr217) agent is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents that substantially optimize the sensitivity of the assay. Particularly, the reagents may be provided as dry powders, usually lyophilized, including excipients that on dissolution will provide a reagent solution having the appropriate concentration.

### Articles of Manufacture

Provided herein are articles of manufacture containing materials useful for the treatment, or diagnosis, of the disorders described above. An article of manufacture may comprise a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active anti-Tau phospho (Thr217) agent in the composition may be an anti-Tau phospho (Thr217) antibody. The label on, or associated with, the container indicates that the composition is used for treating, or diagnosing, the condition of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Certain Implementations

Following are non-limiting examples of certain implementations of the technology.
A1. An isolated antibody or antigen binding fragment thereof that binds tubulin associated unit phosphorylated at threonine 217 (Tau phospho (Thr217)) or a portion thereof, comprising:
   (a) an immunoglobulin heavy chain variable domain comprising:
      (i) a heavy chain complementarity determining region 1 (CDRH1) comprising the polypeptide GYTFTTYG (SEQ ID NO: 4), or a polypeptide that is at least 80 percent identical to the polypeptide GYTFTTYG (SEQ ID NO: 4);
      (ii) a heavy chain complementarity determining region 2 (CDRH2) comprising the polypeptide INTYSGVP (SEQ ID NO: 5), or a polypeptide that is at least 80 percent identical to the polypeptide INTYSGVP (SEQ ID NO: 5); and
      (iii) a heavy chain complementarity determining region 3 (CDRH3) comprising the polypeptide ARTYGERRDYSMDY (SEQ ID NO: 6), or a polypeptide that is at least 80 percent identical to the polypeptide ARTYGERRDYSMDY (SEQ ID NO: 6); and
   (b) an immunoglobulin light chain variable domain comprising:
      (i) a light chain complementarity determining region 1 (CDRL1) comprising the polypeptide ESVEYYGTSL (SEQ ID NO: 7), or a polypeptide that is at least 80 percent identical to the polypeptide ESVEYYGTSL (SEQ ID NO: 7);
      (ii) a light chain complementarity determining region 2 (CDRL2) comprising the polypeptide AAS, or a polypeptide that is at least 80 percent identical to the polypeptide AAS; and
      (iii) a light chain complementarity determining region 3 (CDRL3) comprising the polypeptide QQSRKVPWT (SEQ ID NO: 9), or a polypeptide that is at least 80 percent identical to the polypeptide QQSRKVPWT (SEQ ID NO: 9).
A2. The isolated antibody or antigen binding fragment thereof of embodiment A1, wherein the immunoglobulin heavy chain variable domain comprises the polypeptide of SEQ ID NO: 2, or a polypeptide that is at least 80 percent identical to the polypeptide of SEQ ID NO: 2.
A3. The isolated antibody or antigen binding fragment thereof of embodiment A1 or A2, wherein the immunoglobulin light chain variable domain comprises the polypeptide of SEQ ID NO: 3, or a polypeptide that is at least 80 percent identical to the polypeptide of SEQ ID NO: 3.
A4. The isolated antibody or antigen binding fragment thereof of any one of embodiments A1-A3, comprising two immunoglobulin heavy chain variable domains and two immunoglobulin light chain variable domains.
A5. The isolated antibody or antigen binding fragment thereof of any one of embodiments A1-A4, further comprising one or more human framework regions.
A6. The isolated antibody or antigen binding fragment thereof of any one of embodiments A1-A5, wherein the antibody or antigen binding fragment thereof is humanized.
A7. The isolated antibody or antigen binding fragment thereof of any one of embodiments A1-A6, wherein the antibody or antigen binding fragment thereof comprises or is conjugated to a detectable marker or label.
A8. The isolated antibody or antigen binding fragment thereof of embodiment A7, wherein the detectable marker or label comprises a detectable moiety.
A9. The isolated antibody or antigen binding fragment thereof of any one of embodiments A1-A8, further comprising an oligonucleotide.
A10. The isolated antibody or antigen binding fragment thereof of any one of embodiments A1-A9, wherein the antibody or antigen binding fragment thereof is immobilized on a solid support.
A11. The isolated antibody or antigen binding fragment thereof of any one of embodiments A1-A10, wherein the antibody or antigen binding fragment thereof is non-diffusively immobilized on a solid support.
A12. The isolated antibody or antigen binding fragment thereof of any one of embodiments A1-A11, that is a single-chain fragment.
A13. The isolated antibody or antigen binding fragment thereof of embodiment A12, wherein the single-chain fragment is a single-chain variable fragment (scFv).
A14. The isolated antibody or antigen binding fragment thereof of any one of embodiments A1-A13, for non-diagnostic use and/or non-therapeutic use.
B1. A kit comprising the isolated antibody or antigen binding fragment thereof of any one of embodiments A1-A14, and instructions for use.
C1. A diagnostic reagent comprising the isolated antibody or antigen binding fragment thereof of any one of embodiments A1-A13.
D1. A diagnostic kit configured to detect Tau phospho (Thr217) or a portion thereof in a biological sample, wherein the kit comprises the isolated antibody or antigen binding fragment thereof of any one of embodiments A1-A13 or the diagnostic reagent of embodiment C1.
E1. A therapeutic composition comprising the isolated antibody or antigen binding fragment thereof of any one of embodiments A1-A13, and a pharmaceutically acceptable excipient.
E2. The therapeutic composition of embodiment E1, wherein the antibody or antigen binding fragment thereof is used as an adjuvant or in conjunction with an adjuvant.
F1. An isolated polynucleotide comprising a nucleic acid sequence that encodes the immunoglobulin heavy chain variable domain of any one of embodiments A1-A13.
F2. A recombinant expression vector comprising the isolated polynucleotide of embodiment F1.
F3. A host cell transfected with the recombinant expression vector of embodiment F2.
F4. An isolated polynucleotide comprising a nucleic acid sequence that encodes the immunoglobulin light chain variable domain of any one of embodiments A1-A13.
F5. A recombinant expression vector comprising the isolated polynucleotide of embodiment F4.
F6. A host cell transfected with the recombinant expression vector of embodiment F5.
F7. A recombinant expression vector comprising a first expression cassette and a second expression cassette, wherein the first expression cassette comprises a polynucleotide comprising a nucleic acid sequence that encodes the immunoglobulin heavy chain variable domain of any one of embodiments A1-A13, and the second expression cassette comprises a polynucleotide comprising a nucleic acid sequence that encodes the immunoglobulin light chain variable domain of any one of embodiments A1-A13.
F8. The recombinant expression vector of embodiment F7, wherein the first expression cassette and the second expression cassette each comprise a promoter.
F9. A host cell transfected with the recombinant expression vector of embodiment F7 or F8.
G1. A method of detecting Tau phospho (Thr217), comprising
   a) contacting a sample with the antibody or antigen binding fragment thereof of any one of embodiments A1-A13, and
   b) if the sample contains Tau phospho (Thr217), detecting Tau phospho (Thr217): anti-Tau phospho (Thr217) complexes.
G2. The method of embodiment G1, wherein the method is performed in vitro.
G3. The method of embodiment G1 or G2, wherein the sample is a biological sample.
G4. The method of embodiment G1 or G2, wherein the sample is a non-biological sample.
H1. A first anti-Tau phospho (Thr217) agent that binds Tau phospho (Thr217), wherein the first agent competitively binds, or is capable of competitively binding, with a second anti-Tau phospho (Thr217) agent, wherein the second agent is the antibody or antigen binding fragment thereof of any one of embodiments A1-A13.
H2. A first anti-Tau phospho (Thr217) agent that binds Tau phospho (Thr217), wherein the first agent binds to, or is capable of binding to, the same epitope as a second anti-Tau phospho (Thr217) agent, wherein the second agent is the antibody or antigen binding fragment thereof of any one of embodiments A1-A13.

### EXAMPLES

The examples set forth below illustrate certain implementations and do not limit the technology.

### Example 1: Generation and Evaluation of Tau Phospho (Thr217) Antibodies

This Example describes the generation and evaluation of Tau phospho (Thr217) antibodies. Briefly, Tau phospho (Thr217) antibodies were developed following mouse immunization with human synthetic peptide of Tau phosphorylated at threonine 217 (Tau phospho (Thr217)) and were characterized for specificity, affinity, and selectivity in a variety of applications that may be used to develop diagnostic tests, for example. These antibodies were evaluated in the following applications: 1) Direct ELISA: the antibodies preferentially bind to the synthetic biotinylated human Tau peptide phosphorylated at Thr217 but not its non-phosphorylated analog; 2) Western blot (WB): the antibodies detect phosphorylated Tau isoforms by WB in lysates from rat brains that differentially express Tau (Thr217) phosphorylation based on their age; 3) Immunohistochemistry (IHC): the antibodies detect amyloid plaques in Alzheimer's disease (AD) patient brain; 4) Sandwich ELISA: the antibodies recognize phosphorylated Tau at Thr217 in human brain lysates. The limit of detection of these antibodies in an immunoassay format generally should be at or above around 0.07 pg/ml for detection of Tau phospho (Thr217) in plasma samples. This Example shows these antibodies work in multiple applications such as WB, IHC, indirect and sandwich ELISA, and in terms of quality they are as good or better than existing commercial products. Accordingly, the antibodies provided herein can be used to develop sensitive assays for detecting Tau phospho (Thr217) in biological fluids, for example.

Immunization of IR6 Mice with Phospho and Non-Phospho Tau (Thr217) Peptides and Clone Summary

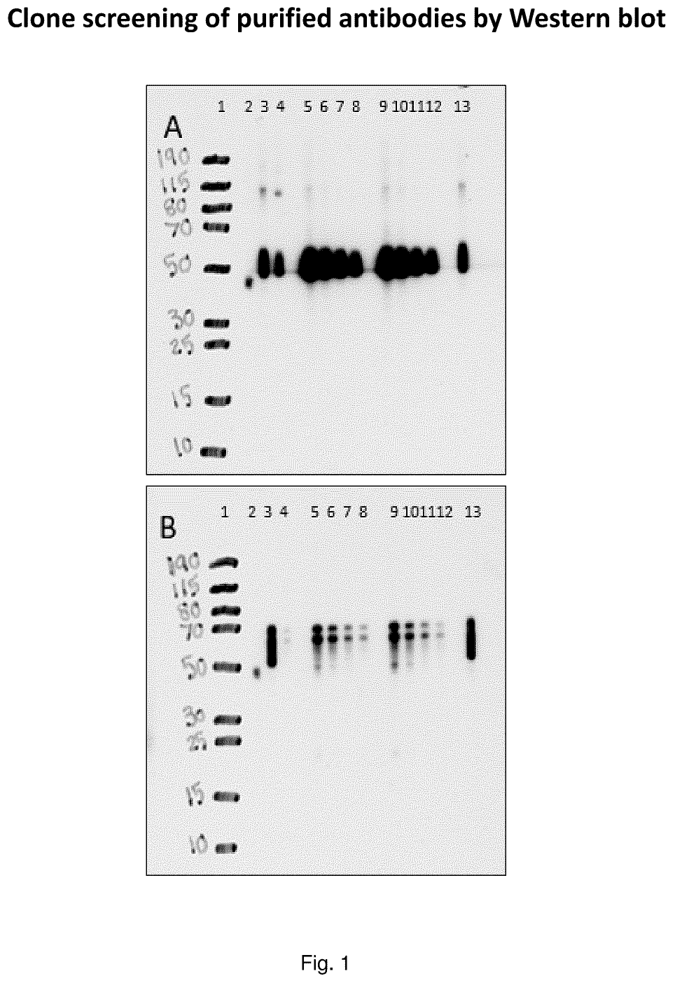

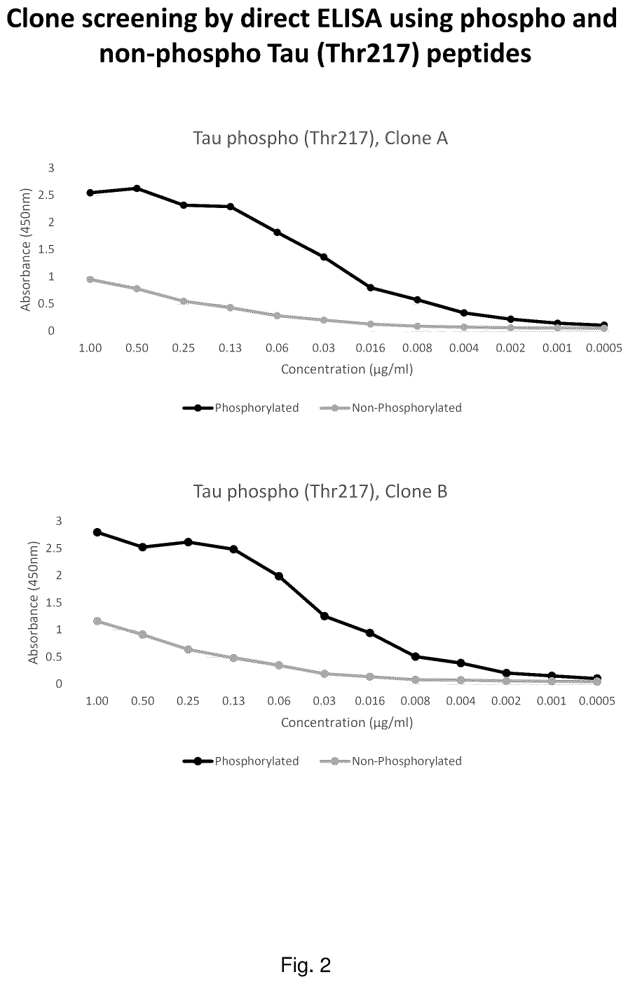

IR6 mice were immunized using a synthetic peptide with the sequence from human Tau phospho (Thr217) of isoforms 5 & 8. The sequence used was: RTPSLP(pT)PPTREPK (SEQ ID NO: 10). A mix of peptide conjugated to KLH carrier protein and Complete Freund's Adjuvant was used for the first immunization and Incomplete Freund's Adjuvant plus peptide for the subsequent immunizations. Each immunization was administered at 2-week intervals. Serum was tested by Western blot (WB) and ELISA after the fifth immunization. The spleen from the mouse with the highest response in both ELISA and WB was fused with mouse myeloma cells, SP2/0 cells, by using the electrofusion (BTX, ECM 2001+) method. The resulting hybridomas were subcloned two times to ensure monoclonality and tested by WB and ELISA at primary, first and second subcloning. The final clones were purified and tested by WB and ELISA at the R&D level. R&D data of the final purified Clone A and Clone B by WB and ELISA is shown in FIG. 1 and FIG. 2, respectively.

FIG. 1 shows clone screening of purified antibodies by Western blot (WB). Whole cell extracts from rat brain (day 2 post birth) (panel A) or rat brain (day 30 post birth) (panel B) were resolved on a 4-12% bis-tris gel and transferred to a PVDF membrane with the molecular weight markers in lane 1. Lanes were immunoblotted as follows: Lane 2: anti-Actin (BD #612656 at 1:3,000). Lane 3: commercial anti-Tau pan antibody at 1:10,000. Lane 4: anti-Tau phospho (Thr217) commercial benchmark antibody (AB1) control at 1:7,500. Lanes 5-8: anti-Tau phospho (Thr217) Clone A at 1 μg/ml, 0.5 μg/ml, 0.25 μg/ml, and 0.125 μg/ml. Lanes 9-12: anti-Tau phospho (Thr217) Clone B at 1 μg/ml, 0.5 μg/ml, 0.25 μg/ml, and 0.125 μg/ml. Lane 13: commercial pan anti-Tau antibody at 1:10,000. Antibodies were incubated for 1 hour at 37° C. Proteins were visualized by chemiluminescence detection using HRP Goat anti-mouse IgG (Jackson Immunoresearch Labs #115-035-164) followed by Western-Ready™ ECL Substrate Plus Kit (Cat. No. 426317) as a detection agent. Both clones recognized endogenous Tau phospho (Thr217) by WB (rat Tau T217 sequence is homologous between human, mouse and rat).

FIG. 2 shows clone screening by direct ELISA using biotin-conjugated phosphorylated and non-phosphorylated Tau (Thr217) peptides (RTPSLP (PT) PPTREPK (SEQ ID NO: 10) and (RTPSLPTPPTREPK (SEQ ID NO: 11)). Direct ELISA of anti-Tau phospho (Thr217) antibody (Clone A, top plot; and Clone B, bottom plot) was performed on biotin conjugated phosphorylated and non-phosphorylated linear peptides immobilized on NeutrAvidin™ Biotin Binding Protein (Thermo #31000) coated plates. The purified anti-Tau Phospho (Thr217) was incubated for 1 hour at room temperature followed by incubation with horseradish peroxidase labeled goat anti-mouse secondary antibody (Jackson Immunoresearch Labs #115-035-164). 3,3′,5,5′ tetramethylbenzidine, TMB (Cat. No. 421501) was used as the detection system. Both clones bound preferentially to the immunogen compared to its non-phosphorylated analog.

Amino acid sequences for Clone A are provided in Table 2 below.

TABLE 2
Clone A Amino Acid Sequences

| | | |
|---|---|---|
| Heavy chain | QIQLVQSGPELKNPGETVTISCKASGYTFTTYGLTWVKQAPGKGLKW | SEQ ID NO: 2 |
| (fwr1-fwr4) | MGWINTYSGVPTHADDFTGRFAFSLETSASTAYLQIDNLNNEDTATYF | |
| | CARTYGERRDYSMDYWGQGTSVTVSS | |
| Light chain | DIVFTQSPASLAVSLGQRATISCRASESVEYYGTSLMQWFQQKPGQP | SEQ ID NO: 3 |
| (fwr1-fwr4) | PKLLIYAASNVESGVPARFSGSGSGTDFSLNIHPVEEDDMAMYFCQQS | |
| | RKVPWTFGGGTKLEIK | |
| CDRH1 | GYTFTTYG | SEQ ID NO: 4 |
| CDRH2 | INTYSGVP | SEQ ID NO: 5 |
| CDRH3 | ARTYGERRDYSMDY | SEQ ID NO: 6 |
| CDRL1 | ESVEYYGTSL | SEQ ID NO: 7 |
| CDRL2 | AAS | |
| CDRL3 | QQSRKVPWT | SEQ ID NO: 9 |
| Heavy chain | QIQLVQSGPELKNPGETVTISCKAS | SEQ ID NO: 12 |
| fwr1 | | |
| Heavy chain | LTWVKQAPGKGLKWMGW | SEQ ID NO: 13 |
| fwr2 | | |
| Heavy chain | THADDFTGRFAFSLETSASTAYLQIDNLNNEDTATYFC | SEQ ID NO: 14 |
| fwr3 | | |
| Heavy chain | WGQGTSVTVSS | SEQ ID NO: 15 |
| fwr4 | | |
| Light chain | DIVFTQSPASLAVSLGQRATISCRAS | SEQ ID NO: 16 |
| fwr1 | | |
| Light chain | MQWFQQKPGQPPKLLIY | SEQ ID NO: 17 |
| fwr2 | | |
| Light chain | NVESGVPARFSGSGSGTDFSLNIHPVEEDDMAMYFC | SEQ ID NO: 18 |
| fwr3 | | |
| Light chain | FGGGTKLEIK | SEQ ID NO: 19 |
| fwr4 | | |

Amino acid sequences for Clone B are provided in Table 3 below.

TABLE 3
Clone B Amino Acid Sequences

| | | |
|---|---|---|
| Heavy chain | QIQLVQSGPELKNPGETVTISCKASGYTFTTYGLTWVKQAPGKGLKW | SEQ ID NO: 2 |
| (fwr1-fwr4) | MGWINTYSGVPTHADDFTGRFAFSLETSASTAYLQIDNLNNEDTATYF | |
| | CARTYGERRDYSMDYWGQGTSVTVSS | |
| Light chain | DIVFTQSPASLAVSLGQRATISCRASESVEYYGTSLMQWFQQKPGQP | SEQ ID NO: 3 |
| (fwr1-fwr4) | PKLLIYAASNVESGVPARFSGSGSGTDFSLNIHPVEEDDMAMYFCQQS | |
| | RKVPWTFGGGTKLEIK | |
| CDRH1 | GYTFTTYG | SEQ ID NO: 4 |
| CDRH2 | INTYSGVP | SEQ ID NO: 5 |
| CDRH3 | ARTYGERRDYSMDY | SEQ ID NO: 6 |
| CDRL1 | ESVEYYGTSL | SEQ ID NO: 7 |
| CDRL2 | AAS | |
| CDRL3 | QQSRKVPWT | SEQ ID NO: 9 |
| Heavy chain | QIQLVQSGPELKNPGETVTISCKAS | SEQ ID NO: 12 |
| fwr1 | | |
| Heavy chain | LTWVKQAPGKGLKWMGW | SEQ ID NO: 13 |
| fwr2 | | |
| Heavy chain | THADDFTGRFAFSLETSASTAYLQIDNLNNEDTATYFC | SEQ ID NO: 14 |
| fwr3 | | |
| Heavy chain | WGQGTSVTVSS | SEQ ID NO: 15 |
| fwr4 | | |
| Light chain | DIVFTQSPASLAVSLGQRATISCRAS | SEQ ID NO: 16 |
| fwr1 | | |
| Light chain | MQWFQQKPGQPPKLLIY | SEQ ID NO: 17 |
| fwr2 | | |
| Light chain | NVESGVPARFSGSGSGTDFSLNIHPVEEDDMAMYFC | SEQ ID NO: 18 |
| fwr3 | | |
| Light chain | FGGGTKLEIK | SEQ ID NO: 19 |
| fwr4 | | |

### Specificity

Three experiments were performed to demonstrate antibody specificity of the Tau phospho (Thr217) antibodies provided herein.

In the first experiment, Clone A and Clone B reacted with the phosphorylated Tau (Thr217) peptide (RTPSLP (PT) PPTREPK (SEQ ID NO: 10)) and not with the non-phosphorylated peptide (RTPSLPTPPTREPK (SEQ ID NO: 11)) in a direct ELISA (FIG. 2; discussed above).

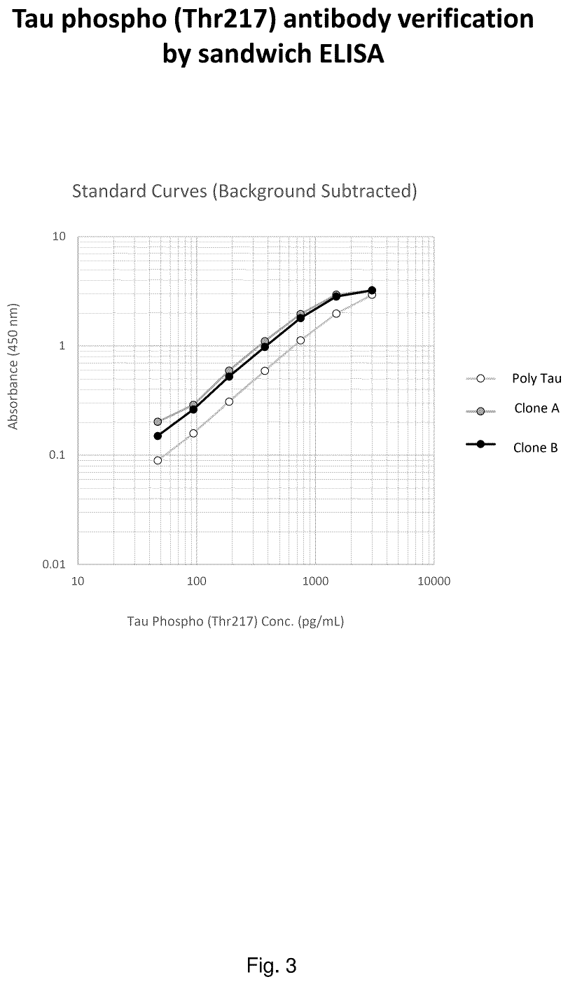

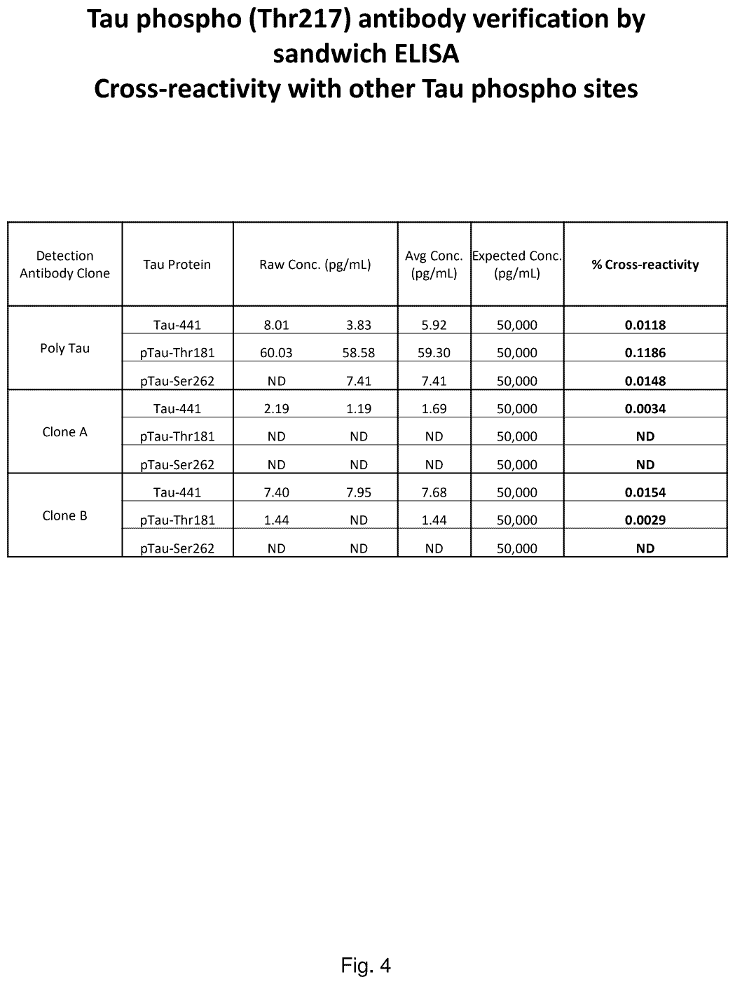

In the second group of experiments, a sandwich ELISA was performed to determine whether the Tau phospho (Thr217) clones provided herein can recognize the phosphorylated site Thr217 using clone TAU-13 (anti-total tau AB) as a capture and Clone A, Clone B, or a poly Tau phospho (Thr217) clone as detectors (data provided in FIG. 3). Cross reactivity with other Tau phospho sites also was evaluated (data provided in FIG. 4).

Sandwich ELISA materials included the following:
a. Nunc™ Maxisorp™ ELISA plate (96-well)
b. 5× Coating Buffer A (diluted to 1× in DI water)
c. Capture antibody (Clone TAU-13)
d. StabilBlock® Immunoassay Stabilizer
e. Assay Diluent B
f. Phospho-Tau (Thr217) standard (Thr217 phospho-peptide conjugated to Tau-441 via a sulfo-SMCC linker)
g. Detection antibodies (Clone A, Clone B, and a poly Tau (T217) clone)
h. Streptavidin-Polymer HRP
i. Substrate Solution F
j. Stop Solution
k. 20× Wash Buffer (diluted to 1× in DI water)

The following ELISA protocol was used:

One day prior to running the ELISA, a 1× stock of Coating Buffer A was prepared by diluting 5× Coating Buffer A in DI water (FIG. 3); Coating Buffer B was used in FIG. 4. Then, the capture antibody was diluted in 1× Coating Buffer A (or B) to a final concentration of 4 μg/mL. A Nunc™ Maxisorp™ ELISA plate (FIG. 3) or Costar® Stripwell™ Microplate (FIG. 4) was coated with 100 μL/well capture antibody and was incubated at 4° C. for 16-18 h. The next day, the plate was washed 4× with 1× Wash Buffer (prepared from a 20× stock diluted in DI water). The plate was then blocked with 200 μL/well StabilBlock® Immunoassay Stabilizer for one hour on a plate shaker (500 rpm). While the plate was blocking, seven two-fold dilutions of the Tau phospho (Thr217) standard were prepared in Assay Diluent B (FIG. 3) or Assay Buffer B (FIG. 4), with buffer alone serving as the background control. The top standard value was 1500 μg/mL (FIG. 3) or 3000 μg/mL (FIG. 4). The blocked plate was washed 4× with 1× Wash Buffer, then 100 μL/well of the diluted standards were added (FIG. 3). For FIG. 4, 50 μL/well of the samples were added to 50 μL/well Assay Buffer B. The plate was incubated for two hours with shaking. Then, detection antibodies were prepared in Assay Diluent B (0.5 g/mL for each antibody) (FIG. 3) or in Assay Buffer B (0.3 μg/mL for each antibody) (FIG. 4). The plate was washed after two hours, then incubated with 100 μL/well of the detection antibodies for one hour with shaking. The streptavidin-polymer HRP was prepared to a final concentration of 0.2 μg/mL in StabilZyme™ HRP Stabilizer (FIG. 3) or 0.4 μg/mL in MossGuard™-M HRP Stabilizing Diluent (FIG. 4). After one hour, the plate was washed and then incubated with 100 μL/well of streptavidin-polymer HRP for 30 minutes with shaking. Afterwards, the plate was washed 5×; each individual wash was at least 30 seconds to allow removal of excess streptavidin-polymer HRP. Then, 100 μL/well Substrate Solution F was added to the plate and incubated for 10 minutes in the dark. The solution began to turn blue, with the intensity of the color being proportional to the amount of Tau phospho (Thr217) in the sample. After 10 minutes, 100 μL/well Stop Solution was added to end the reaction; the color changed from blue to yellow. The absorbance of each well was read in a microplate reader at 450 nm (the background absorbance measured at 570 nm was subtracted).

FIG. 3 shows performance of Clone A and Clone B in the sandwich ELISA compared to a polyclonal antibody against the same specificity. The concentration of each standard curve dilution was plotted against the corresponding absorbance. The standard curve ranges from 46.9 to 3000 μg/mL. Both Clone A and Clone B performed as well as or better than the polyclonal antibody (benchmark) with similar sensitivity under this assay configuration.

FIG. 4 shows cross-reactivity with other Tau phospho sites. Unconjugated full-length Tau (Tau-441) and two Tau phospho-proteins (prepared by conjugating full-length Tau to Tau phospho (Thr181) or Tau phospho (Ser262) peptides) were tested at 50 ng/mL in the Tau phospho (Thr217) ELISA. The percentage of cross-reactivity was calculated for each protein when tested using TAU-13 (capture antibody) and poly Tau phospho (Thr217) clone, Clone A, or Clone B for detection. The anti-Tau phospho (Thr217) did not cross react with phosphorylated Tau (Thr181) or Tau (Ser262), indicating that Clone A and Clone B are both specific to the phosphorylated Thr217 site and selective (are not cross reactive with total Tau or an irrelevant Tau phospho site).

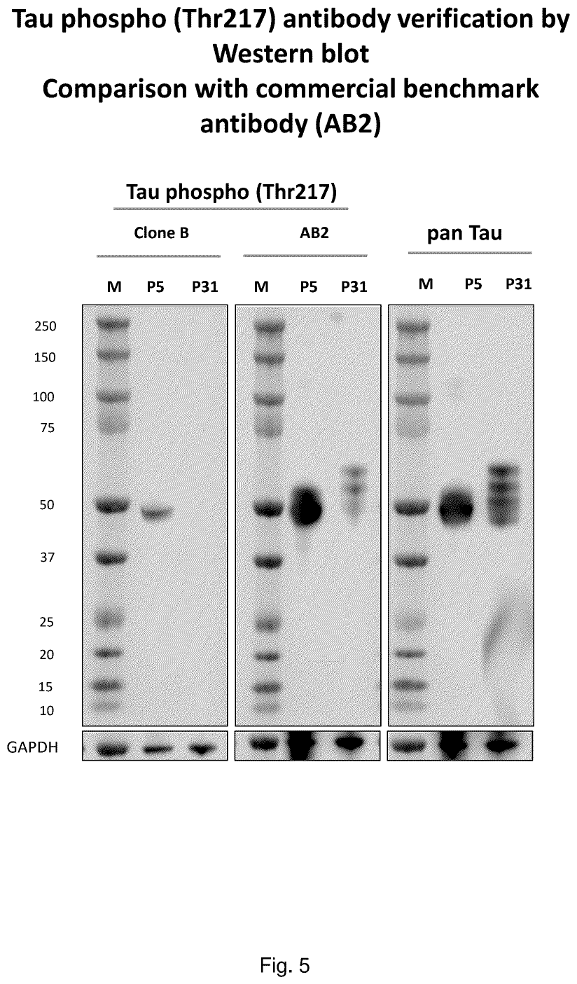

In the third group of experiments, Clone A and Clone B were evaluated by Western blot (WB) using purified anti-Tau phospho (Thr217). FIG. 5 shows Tau phospho (Thr217) antibody verification by Western blot (comparison with commercial benchmark control (AB2)). Whole cell extracts (15 μg total protein) from 5 days old (positive control, P5) or 31 days old (negative control, P31) rat brains were resolved on a 4-12% bis-tris gel, transferred to a PVDF membrane and then probed with purified anti-Tau phospho (Thr217) (Clone B) (left panel), purified anti-Tau phospho (Thr217) commercial benchmark antibody (AB2; middle panel) or purified commercial pan anti-Tau antibody (right panel). Proteins were visualized by chemiluminescence detection using HRP goat anti-mouse IgG (Cat. No. 405306) or HRP Donkey anti-rabbit (Cat. No. 406401). Direct-Blot™ HRP anti-GAPDH (Cat. No. 607904) was used as a loading control. Western-Ready™ ECL Substrate Premium Kit (Cat. No. 426318) was used as a detection agent. Lane M: molecular weight marker. FIG. 5 only shows Clone B but is representative of both Clone A and Clone B. The commercial benchmark control (AB2) showed some pan reactivity.

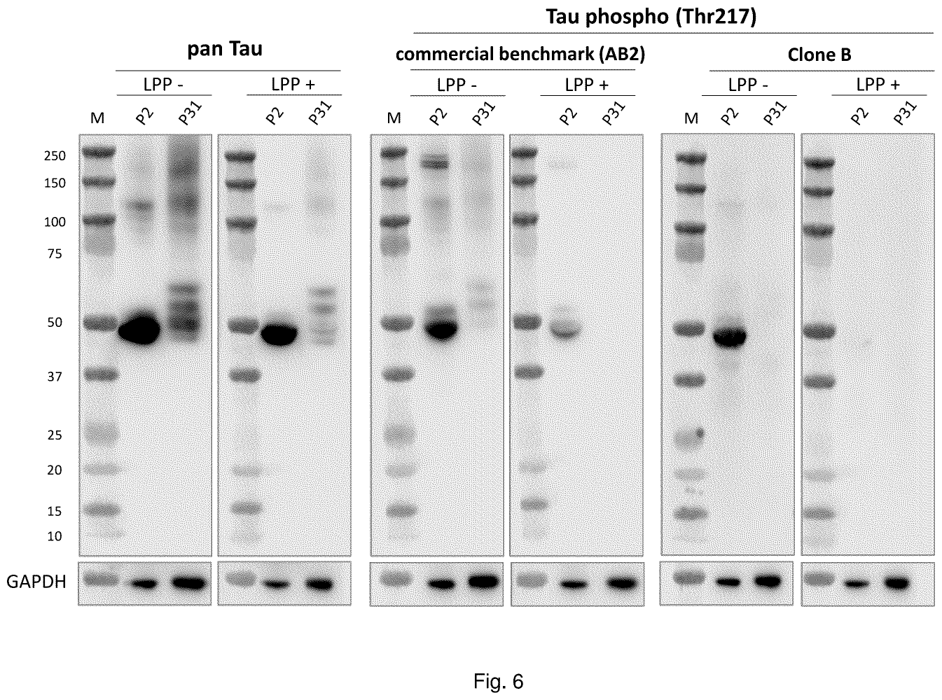

Phospho-specificity in WB analysis was demonstrated with phosphatase treatment. FIG. 6 shows Tau phospho (Thr217) antibody verification by Western blot (comparison with commercial benchmark control (AB2)). Whole cell extracts (15 μg total protein) from 2 days old (positive control, P2) or 31 days old (negative control, P31) rat brains were resolved on a 4-12% bis-tris gel, transferred to a PVDF membrane and then treated with Lambda protein phosphatase (LPP+). The blots were probed with purified anti-Tau phospho (Thr217) (Clone B), purified anti-Tau phospho (Thr217) commercial benchmark antibody (AB2) or purified commercial pan anti-Tau antibody. Proteins were visualized by chemiluminescence detection using HRP goat anti-mouse IgG (Cat. No. 405306) or HRP Donkey anti-rabbit (Cat. No. 406401). Direct-Blot™ HRP anti-GAPDH (Cat. No. 607904) was used as a loading control. Western-Ready™ ECL Substrate Premium Kit (Cat. No. 426318) was used as a detection agent. Lane M: Molecular weight marker. FIG. 6 only shows Clone B but is representative of both Clone A and Clone B. The commercial benchmark control (AB2) showed residual binding to the target following phosphatase treatment suggesting it may not be exclusively phospho-specific and may partially recognize total Tau.

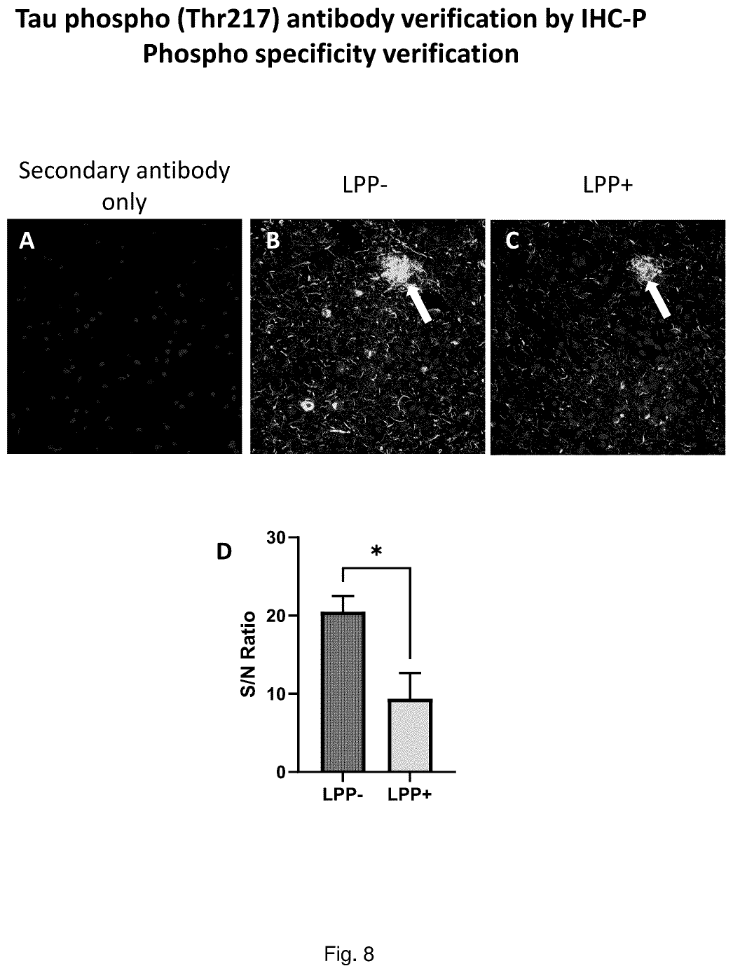

Phospho-specificity in IHC analysis was demonstrated with phosphatase treatment. FIG. 8 shows Tau phospho (Thr217) antibody verification by IHC-P (phospho specificity verification). IHC staining of Tau phospho (Thr217) was performed on formalin-fixed paraffin-embedded (FFPE) human Alzheimer's disease cortex untreated (positive control, LPP−; panel B) or treated with Lambda protein phosphatase (LPP) (negative control, LPP+; panel C). The tissue sections were incubated with Alexa Fluor® 647 secondary antibody only (panel A) or with purified anti-Tau phospho (Thr217) (Clone B; panels B and C) followed by Alexa Fluor® 647 goat anti-mouse (Cat. No. 405322). Nuclei were counterstained with DAPI (Cat. No. 422801). The staining on amyloid plaques and neurofibrillary tangles on human Alzheimer's FFPE tissue was greatly reduced following phosphatase treatment (white arrows). Panel D represents the signal to noise ratio (S/N ratio) (mean±SEM) of LPP untreated tissue (LPP-) versus LPP treated tissue (LPP+) (*: p=0.0207).

Clone B recognized a phosphorylated epitope by both WB (FIG. 6) and IHC (FIG. 8). Phosphatase treatment of the PVDF membrane and the tissue section, respectively, prior to staining with the anti-Tau phospho (Thr217) clone eliminated or greatly reduced antibody binding.

### Antibody Affinity

An experiment using a sandwich ELISA described above was performed to demonstrate affinity of the Tau phospho (Thr217) antibodies provided herein. Performance of Clone A and Clone B in a sandwich ELISA was compared to a polyclonal antibody (poly Tau phospho (Thr217)) against the same specificity. Serial dilutions of a Tau phospho (Thr217) standard were tested in a sandwich ELISA utilizing the following antibody pairs: Clone TAU-13 (capture antibody) with either poly Tau phospho (Thr217), Clone A, or Clone B for detection. The concentration of each standard curve dilution was plotted against the corresponding absorbance (FIG. 3; OD measured at 450 nm). The standard curve ranges from 46.9 to 3000 μg/mL. Clone A and Clone B clones outperformed the poly Tau phospho (Thr217) antibody.

### Selectivity

Two experiments were performed to demonstrate affinity of the Tau phospho (Thr217) antibodies provided herein.

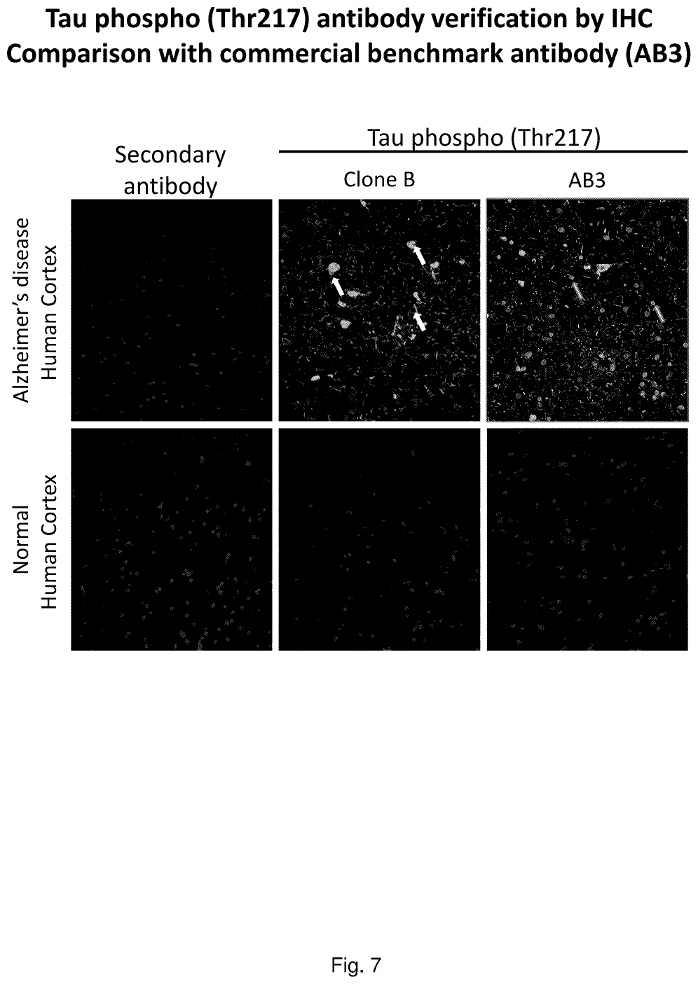

In the first experiment, IHC-P was performed for Clone A and Clone B and compared to commercial benchmark antibody (AB3)). IHC staining of Tau phospho (Thr217) was performed on FFPE human Alzheimer's disease cortex (positive control; FIG. 7, top panels) or human normal cortex (negative control; FIG. 7, bottom panels) following antigen retrieval using Citrate buffer (Cat. No. 420901). The tissue sections were incubated with Alexa Fluor® 647 secondary antibody only (FIG. 7, left panels) or with purified anti-Tau phospho (Thr217) (Clone B; FIG. 7, middle panels), or with purified commercial anti-Tau phospho (Thr217) (FIG. 7, right panels) followed by Alexa Fluor® 647 goat anti-mouse (Cat. No. 405322), or Alexa Fluor® 647 donkey anti-rabbit (Cat. No. 406414). Nuclei were counterstained with DAPI (Cat. No. 422801). FIG. 7 shows Clone B recognized amyloid plaques (upper arrows) and neurofibrillary tangles (lower arrow) in FFPE Alzheimer's human cortex tissue. This was compared to commercial benchmark antibody (AB3). The benchmark antibody showed background nuclear staining indicated with arrows. Clone B demonstrated better selectivity compared to its counterpart (commercial benchmark control (AB3)). The commercial benchmark control (AB3) showed non-specific nuclear staining. FIG. 7 only shows Clone B but is representative of both Clone A and Clone B.

In the second experiment, WB was performed as described above. Phosphatase treatment of the PVDF membrane completely abrogated binding of Clone B but the commercial antibody (AB2) still bound to the membrane indicating some nonspecific reactivity with total Tau (FIG. 6). Thus, Clone B was more selective than commercial antibody (AB2). In addition, FIG. 6 shows Clone B had no background in WB in the absence of phosphatase treatment.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. An isolated antibody or antigen binding fragment thereof that binds tubulin associated unit phosphorylated at threonine 217 (Tau phospho (Thr217)) or a portion thereof, comprising:
(a) an immunoglobulin heavy chain variable domain comprising:
(i) a heavy chain complementarity determining region 1 (CDRH1) comprising the polypeptide GYTFTTYG (SEQ ID NO: 4);
(ii) a heavy chain complementarity determining region 2 (CDRH2) comprising the polypeptide INTYSGVP (SEQ ID NO: 5); and
(iii) a heavy chain complementarity determining region 3 (CD RH3) comprising the polypeptide ARTYGERRDYSMDY (SEQ ID NO: 6); and
(b) an immunoglobulin light chain variable domain comprising:
(i) a light chain complementarity determining region 1 (CDRL1) comprising the polypeptide ESVEYYGTSL (SEQ ID NO: 7);
(ii) a light chain complementarity determining region 2 (CDRL2) comprising the polypeptide AAS; and
(iii) a light chain complementarity determining region 3 (CDRL3) comprising the polypeptide QQSRKVPWT (SEQ ID NO: 9).

2. The isolated antibody or antigen binding fragment thereof of claim 1, comprising two immunoglobulin heavy chain variable domains and two immunoglobulin light chain variable domains.

3. The isolated antibody or antigen binding fragment thereof of claim 2, wherein the immunoglobulin heavy chain variable domain comprises the polypeptide of SEQ ID NO: 2, or a polypeptide that is at least 80 percent identical to the polypeptide of SEQ ID NO: 2; and the immunoglobulin light chain variable domain comprises the polypeptide of SEQ ID NO: 3, or a polypeptide that is at least 80 percent identical to the polypeptide of SEQ ID NO: 3.

4. A diagnostic antibody or antigen binding fragment thereof comprising the antibody or antigen binding fragment thereof of claim 1.

5. A kit comprising the antibody or antigen binding fragment thereof of claim 1.

6. A pharmaceutical composition comprising the antibody or antigen binding fragment thereof of claim 1, and a pharmaceutically acceptable excipient.

7. An isolated nucleic acid comprising a nucleotide sequence that encodes the immunoglobulin heavy chain variable domain of the antibody or antigen binding fragment thereof of claim 1.

8. A recombinant expression vector comprising the isolated nucleic acid of claim 7.

9. A host cell comprising the recombinant expression vector of claim 8.

10. An isolated nucleic acid comprising a nucleotide sequence that encodes the immunoglobulin light chain variable domain of the antibody or antigen binding fragment thereof of claim 1.

11. A recombinant expression vector comprising the isolated nucleic acid of claim 10.

12. A host cell comprising the recombinant expression vector of claim 11.

13. A recombinant expression vector comprising a first expression cassette and a second expression cassette, wherein the first expression cassette comprises a nucleic acid molecule comprising a nucleotide sequence that encodes the immunoglobulin heavy chain variable domain of claim 1, and the second expression cassette comprises a nucleic acid molecule comprising a nucleotide sequence that encodes an immunoglobulin light chain variable domain of the antibody or antigen binding fragment thereof of claim 1.

14. A host cell comprising the recombinant expression vector of claim 13.

15. A method of detecting Tau phospho (Thr217), the method comprising: contacting a sample with the antibody or antigen binding fragment thereof of claim 1, under conditions to bind the antibody or antigen binding fragment thereof to Tau phospho (Thr217) in the sample, wherein the binding generates the production of a Tau phospho (Thr217)/antibody or antigen binding fragment thereof complex.
